(19) United States
(12) Patent Application Publication
(10) Pub. No.: US 2025/0325521 A1
(43) Pub. Date: Oct. 23, 2025

(54) USE OF SUBLINGUAL DEXMEDETOMIDINE FOR THE TREATMENT OF AGITATION

(71) Applicant: BioXcel Therapeutics, Inc., New Haven, CT (US)

(72) Inventors: Krishnan NANDABALAN, New Haven, CT (US); Frank YOCCA, New Haven, CT (US); Sameer SHARMA, New Haven, CT (US); Harsh NEGI, New Delhi (IN); Deepa SAINI, Ghaziabad (IN)

(21) Appl. No.: 18/967,863

(22) Filed: Dec. 4, 2024

Related U.S. Application Data
(63) Continuation of application No. 18/436,589, filed on Feb. 8, 2024, now abandoned.
Application No. 18/436,589 is a continuation of application No. 17/496,470, filed on Oct. 7, 2021, now Pat. No. 11,931,340.
Application No. 17/496,470 is a continuation of application No. 16/474,882, filed on Jun. 28, 2019, now Pat. No. 11,839,604.
(60) Provisional application No. 62/441,164, filed on Dec. 31, 2016.
(60) Provisional application No. 62/471,393, filed on Mar. 15, 2017.
(60) Provisional application No. 62/542,323, filed on Aug. 8, 2017.

(51) Int. Cl. A61K 31/4174; A61K 9/00; A61K 9/20; A61K 45/06; A61K 47/10

(52) U.S. Cl. CPC A61K 31/4174; A61K 9/006; A61K 9/2027; A61K 45/06; A61K 47/10

(57) ABSTRACT

The present invention discloses a method of treating agitation or the signs of agitation in a subject comprising the sublingual administration of an effective amount of an alpha-2 adrenergic agonist, more particularly Dexmedetomidine, or a pharmaceutically acceptable salt thereof. The method is particularly suitable for the treatment of agitation associated with neurodegenerative and/or neuropsychiatric diseases. The present invention also discloses the sublingual administration of an alpha-2 adrenergic agonist, more particularly Dexmedetomidine or a pharmaceutically acceptable salt thereof at a dose that is effective to treat agitation or the signs of agitation in a subject, but does not cause significant sedation.

### CROSS REFERENCE TO RELATED APPLICATION

This application is a continuation of U.S. patent application Ser. No. 18/436,589, filed Feb. 8, 2024, which is a continuation of U.S. patent application Ser. No. 17/496,470, filed Oct. 7, 2021, now U.S. Pat. No. 11,931,340, which is a continuation of U.S. patent application Ser. No. 16/474,882, filed Jun. 28, 2019, now U.S. Pat. No. 11,839,604, which is a U.S. national stage application of International Patent Application No. PCT/US2017/069030, filed Dec. 29, 2017, which claims the benefit of priority to U.S. Provisional Application No. 62/441,164, filed Dec. 31, 2016, U.S. Provisional Application No. 62/471,393 filed Mar. 15, 2017, and U.S. Provisional Application No. 62/542,323, filed Aug. 8, 2017, the disclosures of which are herein incorporated by reference in their entirety for all purposes.

### FIELD OF THE INVENTION

The present invention discloses a method of treating agitation or the signs of agitation in a subject comprising sublingually administering an effective amount of an alpha-2 adrenergic agonist, more particularly Dexmedetomidine or a pharmaceutically acceptable salt thereof. The present invention also discloses a sublingual composition for treating agitation or the signs of agitation comprising an effective amount of Dexmedetomidine or a pharmaceutically acceptable salt thereof together with one or more pharmaceutically acceptable carriers and/or excipients, along with the preparation thereof.

### BACKGROUND OF THE INVENTION

Agitation is an umbrella term that can refer to a range of behavioral disturbances or disorders, including aggression, combativeness, hyperactivity, and disinhibition. Agitation is a nonspecific constellation of relatively unrelated behaviors that can be seen in several different clinical conditions, usually presenting a fluctuating course. Agitation may be caused by several different medical conditions and drug interactions or by any circumstances that worsen the person's ability to think. Multiple underlying pathophysiologic abnormalities are mediated by dysregulations of dopaminergic, serotonergic, noradrenergic, and GABAergic systems. Agitation is characterized by non-productive, diffuse and excessive over-activity both motor (akathisia) and cognitive, and accompanied by an inner unpleasant tension. The key to safety is to intervene early to prevent progression of agitation to aggression and violence.

Agitation can be associated with neurodegenerative disorders. One of the important manifestations of long-term progressive neurodegenerative process is clinically known as dementia. Dementias include Alzheimer's disease dementia (AD), Fronto-temporal dementia (FTD), Vascular dementia, Lewy body disease (LBD), and Down dementia. Dementia in adults, gradually destroy a person's memory and ability to learn, reason, make judgments, communicate and carry out daily activities. In later stages, patients may experience changes in personality and behavior, such as anxiety, suspicion, agitation and aggression.

Sebastiaan Engelborghs et al., in Neurochemistry International 2007 November, 52(6): 1052-60, disclosed that, in frontotemporal dementia, increased activity of dopaminergic neurotransmission and altered serotonergic modulation of dopaminergic neurotransmission are associated with agitated and aggressive behavior respectively. Pia Jul et al., in Journal of Alzheimer's disease 2015 September, 49(3): 783-95, disclosed that rTg4510 mice exhibited P301L-tau-dependent hyperactivity, and agitation-like phenotypes in these mice may form a correlation to some of the behavioral disturbances observed in advanced Alzheimer's disease (AD) and Frontotemporal dementia (FTD). Nathan Hermann et. al., in Journal of Neuropsychiatry 2004 August, 16(3): 261-276, disclosed that a compensatory increase in activity within the noradrenergic system may contribute to the behavioral and psychological symptoms of agitation and aggression in Alzheimer's disease.

Agitation can also be associated with neuropsychiatric conditions such as schizophrenia, bipolar illness such as bipolar disorder or mania, depression, delirium, etc or agitation can be associated with alcohol and substance abuse withdrawal. Acute agitation, represented by a state of motor restlessness and accompanying mental tension, is a serious medical problem that can be present in some psychiatric disorders, including schizophrenia and bipolar mania, and may escalate quickly to aggressive behavior. Acute agitation is characterized by signs that include pacing, hand wringing, fist clenching, pressured speech, yelling, and threatening people with escalated agitation.

To date, there is no single medication considered as the “standard of care” for treating agitation in patients with dementia or schizophrenia. Generally, three classes of medications are used most frequently, depending on the severity of the agitation, namely first-generation antipsychotics, second-generation antipsychotics, and benzodiazepines, administered orally, intramuscularly or intravenously. Intramuscular injection of typical antipsychotics and benzodiazepines, given alone or in combination, has been a treatment of choice for agitation over the past few decades. The currently preferred treatment paradigm for acute agitation is to use atypical antipsychotic drugs administered with or without supplemental benzodiazepines.

More specifically, patients with agitation are usually prescribed beta blockers such as propranolol and Pindolol, anxiety medications such as Buspirone, benzodiazepines such as Lorazepam, anti-convulsants such as Valproate and Lamotrigine, anti-psychotics such as Haloperidol, Droperidol, Ziprasidone and other high-potency dopamine-blocking agents, and atypical antipsychotics such as Olanzapine. However, Buspirone, Valproate, Haloperidol, Droperidol and Ziprasidone have potential adverse effects, and optimal dosage and long-term efficacy in the management of chronic agitation in dementia is very limited. Lorazepam is only effective for treating agitation in patients when used before medical procedures. Loxapine (an antipsychotic) is FDA approved for treating agitated patients via inhalation, but is associated with a black box warning for bronchospasm and increased mortality in elderly patients with dementia-related psychosis (FDA label, Loxapine or Adasuve®). Olanzapine, Ziprasidone or its combination with Haloperidol, is also associated with QT prolongation, and extrapyramidal side effects should be watched very carefully in hospital set ups. Reports of adverse events (including eight fatalities) associated with intramuscular olanzapine underscores the need to follow strict prescribing guidelines and avoid simultaneous use with other CNS depressants.

The Expert Consensus Guidelines for treatment of behavioral emergencies cite speed of onset as one of the most important factors in choosing a drug and its route of administration. However, antipsychotic medications can take from days to weeks before having a robust antipsychotic effect. Nevertheless, they do generally have a calming effect on agitated patients within minutes. For example, benzodiazepines or fast-acting sedatives quickly calm a severely agitated patient, but continuous treatment with these drugs leads to tolerance.

Therefore, the treatment of agitation in patients with neuropsychiatric conditions (such as schizophrenia or bipolar mania) and neurodegenerative diseases is still limited because of the potential for significant side effects associated with currently used drugs, their route of administration (intravenous/intramuscular) and the consequent need for hospital set ups for administering these drugs. In an ideal situation, an anti-agitation drug for schizophrenics or dementia patient should have a rapid onset of calming without sedation, be well tolerated and easy to administer with a high safety margin.

Alpha-2 adrenergic agonists have been used therapeutically for a number of conditions, including hypertension, congestive heart failure, angina pectoris, spasticity, glaucoma, diarrhea and for suppression of opiate withdrawal symptoms. Examples of alpha-2 adrenergic agonists include Clonidine, Guanfacine, Guanabenz, Guanoxabenz, Guanethidine, Xylazine, Tizanidine, Medetomidine, Dexmedetomidine, Methyldopa, Methylnorepinephrine, Fadolmidine, Iodoclonidine, Apraclonidine, Detomidine, Lofexidine, Amitraz, Mivazerol, Azepexol, Talipexol, Rilmenidine, Naphazoline, Oxymetazoline, Xylometazoline, Tetrahydrozoline, Tramazoline, Talipexole, Romifidine, propylhexedrine, Norfenefrine, Octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione. The inventors of the present invention have unexpectedly found that the sub-lingual administration of an alpha-2 adrenergic agonist or a pharmaceutically acceptable salt thereof is a particularly effective and safe intervention for the treatment of agitation.

(S)-4-[1-(2,3-Dimethylphenyl)ethyl]-3H-imidazole (Dexmedetomidine) is commercially available as an injectable formulation for sedation of initially intubated and mechanically ventilated patients during treatment in an intensive care setting, and for non-intubated patients prior to and/or during surgical and other procedures.

Dexmedetomidine is reported to have anti-agitational effects when administered intravenously or buccally during surgical procedures and intensive care unit (ICU) setups. For example, Ibacache et. al., in Anesthesia & Analgesia 2004 January;98(1):60-3, discloses the administration of an intravenous single-dose of Dexmedetomidine to reduce agitation following sevoflurane anesthesia in children. Other intravenous administrations are reported by Jeanne Boyer et al., in Nursing Critical care 2010 January, 5(1):30-34, Yahya Shehabi et. al., in Anesthetic Intensive Care 2010 January, 38(1):82-90, and Joseph D. Tobias in Journal of Pediatric Pharmacology Therapeutic, January-March 2010, 15(1):43-48. NCT 02720705 (clinical trial identification number from clinicaltrials.gov) discloses the administration of transbuccal Dexmedetomidine for the prevention of emergence agitation in pre-school children treated with sevoflurane in an intensive care unit setting.

The sublingual use of Dexmedetomidine is disclosed in WO 2016/061413. However, the focus of WO 2016/061413 is the administration of Dexmedetomidine sublingually at doses appropriate to treat sleep disorders and induce significant sedation. We have now surprisingly found that Dexmedetomidine or a pharmaceutically acceptable salt thereof, administered sublingually, can effectively treat agitation, including agitation associated with neurodegenerative diseases (e.g.. Alzheimer's disease, fronto-temporal dementia, and sundown syndrome in Alzheimer's disease/dementia), agitation associated with neuropsychiatric conditions (e.g.. bipolar disorder, schizophrenia, bipolar mania, delirium and depression), agitation associated with alcohol and substance abuse withdrawal or agitation associated with other conditions such as OPD/IPD procedures (e.g.. MRI, CT or CAT scan, lumbar puncture, bone marrow aspiration/biopsy, tooth extraction or other dental procedures). The dose to be administered sublingually may be selected to be effective to treat agitation, yet insufficient to causing significant sedation.

### SUMMARY OF THE INVENTION

The present invention provides a method of treating agitation or the signs of agitation in a subject in need thereof, comprising administering an effective amount of an alpha-2 adrenergic agonist or a pharmaceutically acceptable salt thereof sublingually to the subject, wherein the said agitation is associated with a neurodegenerative disease like dementia, Alzheimer's disease, frontotemporal dementia, or Parkinsonism, or associated with a neuropsychiatric condition like schizophrenia, bipolar disorder, bipolar mania, delirium, or depression, or associated with an OPD/IPD procedure (e.g.. MRI, CT or CAT scan, lumbar puncture, bone marrow aspiration/biopsy, tooth extraction or other dental procedures), or associated with an alcohol and substance abuse withdrawal. In a particular aspect, the agitation is suppressed without also causing significant sedation.

In a preferred aspect, the present invention provides a method of treating agitation or the signs of agitation in a subject in need thereof, comprising administering an effective amount of Dexmedetomidine or a pharmaceutically acceptable salt thereof sublingually to the subject. In a particular aspect, the agitation is suppressed without also causing significant sedation.

Another aspect of the present invention provides a method of treating agitation or the signs of agitation in a subject in need thereof, wherein said agitation is associated with neurodegenerative disease, comprising administering an effective amount of Dexmedetomidine or a pharmaceutically acceptable salt thereof sublingually to the subject. In a particular aspect, the agitation is suppressed without also causing significant sedation.

Yet another object of the present invention provides a method of treating agitation or the signs of agitation in a subject in need thereof, wherein said agitation is associated with dementia, Alzheimer's disease, frontotemporal dementia, Parkinsonism or other neurodegenerative diseases, comprising administering an effective amount of Dexmedetomidine or a pharmaceutically acceptable salt thereof sublingually to the subject In a particular aspect, the agitation is suppressed without also causing significant sedation.

Another object of the present invention provides a method of treating agitation or the signs of agitation in a subject in need thereof, wherein said agitation is associated with schizophrenia, bipolar disorder, bipolar mania, delirium, depression, or another related neuropsychiatric condition, comprising administering an effective amount of Dexmedetomidine or a pharmaceutically acceptable salt thereof sublingually to the subject. In a particular aspect, the agitation is suppressed without also causing significant sedation.

A further object of the present invention provides a method of treating, preventing or reducing the signs of agitation in a subject in need thereof, wherein said agitation is associated with sundown syndrome in Alzheimer's disease/dementia, comprising administering an effective amount of Dexmedetomidine or a pharmaceutically acceptable salt thereof sublingually to the subject. In a particular aspect, the agitation is suppressed without also causing significant sedation.

Yet another objective of the present invention provides a method for treating agitation or the signs associated with agitation in a subject in need thereof, wherein said agitation is associated with an OPD/IPD procedure (e.g.., MRI, CT or CAT scan, lumbar puncture, bone marrow aspiration/biopsy, tooth extraction or other dental procedures), comprising administering an effective amount of Dexmedetomidine or a pharmaceutically acceptable salt thereof sublingually to the subject. In a particular aspect, the agitation is suppressed without also causing significant sedation.

Yet another objective of the present invention provides a method for treating agitation or the signs associated with agitation in a subject in need thereof, wherein said agitation is associated with an alcohol and substance abuse withdrawal, comprising administering an effective amount of Dexmedetomidine or a pharmaceutically acceptable salt thereof sublingually to the subject. In a particular aspect, the agitation is suppressed without also causing significant sedation.

A further aspect of the present invention provides a sublingual composition for treating agitation or the signs of agitation in a subject in need thereof, wherein said agitation is associated with a neurodegenerative disease, and said sublingual composition comprises an effective amount of Dexmedetomidine or a pharmaceutically acceptable salt thereof, together with one or more pharmaceutical acceptable carriers and/or excipients.

Another aspect of the present invention provides a sublingual composition for treating agitation or the signs of agitation in a subject in need thereof, wherein said agitation is associated with schizophrenia, bipolar disorder, bipolar mania, delirium, depression, or another related neuropsychiatric condition, and said sublingual composition comprises an effective amount of Dexmedetomidine or a pharmaceutically acceptable salt thereof, together with one or more pharmaceutically acceptable carriers and/or excipients.

An additional aspect of the present invention provides a sublingual composition for treating agitation or the signs of agitation in a subject in need thereof, wherein said agitation is associated with sundown syndrome in Alzheimer's disease/dementia, and said sublingual composition comprises an effective amount of Dexmedetomidine or a pharmaceutically acceptable salt thereof together with one or more pharmaceutically acceptable carriers and/or excipients

Yet another aspect of the present invention provides a sublingual composition for treating agitation or the signs associated with agitation in a subject in need thereof, wherein said agitation is associated with an OPD/IPD procedure (e.g.. MRI, CT or CAT scan, lumbar puncture, bone marrow aspiration/biopsy, tooth extraction or other dental procedures), and said sublingual composition comprises an effective amount of Dexmedetomidine or a pharmaceutically acceptable salt thereof together with one or more pharmaceutically acceptable carriers and/or excipients.

Yet another aspect of the present invention provides a sublingual composition for treating agitation or the signs associated with agitation in a subject in need thereof, wherein said agitation is associated with an alcohol and substance abuse withdrawal, and said sublingual composition comprises an effective amount of Dexmedetomidine or a pharmaceutically acceptable salt thereof together with one or more pharmaceutically acceptable carriers and/or excipients.

Another object of the present invention provides a sublingual composition comprising an effective amount of Dexmedetomidine or a pharmaceutically acceptable salt thereof together with one or more pharmaceutically acceptable carriers and/or excipients, wherein said sublingual composition is selected from the group consisting of a film, wafer, patch, lozenge, gel, spray, tablet, liquid drops or the like.

A further object of the present invention provides a method of sublingually administering an effective amount of Dexmedetomidine or a pharmaceutically acceptable salt thereof to a subject's oral mucosa to treat agitation or the signs of agitation at a dosage which does not cause significant sedation.

In a particular aspect of the invention, the dosage administered sublingually may conveniently be in the range of between about 3 micrograms to about 100 micrograms, Examples of suitable dosages include: about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about $ micrograms to about 15 micrograms, about 5 micrograms to about 10 micrograms, less than 10 micrograms (e.g.. about 5, 6, 7, 8, or 9 micrograms), about 10 micrograms, about 12 micrograms, about 14 micrograms, about 15 micrograms, about 16 micrograms, about 18 micrograms, about 20 micrograms, about 30 micrograms, about 50 micrograms. The dose may be administered one or more times a day.

### BRIEF DESCRIPTION OF THE DRAWINGS

FIG. 1A. Effect of sublingually administered Dexmedetomidine hydrochloride (Dex) at varying doses (0.5-3 μg/kg) on cumulative duration of aggressive and agitated behaviors. Data expressed as Mean±SEM. One-way ANOVA followed by Dunnett's post-hoc test. *p<0.05 **p<0.01, ***p<0.001 and ****p<0.0001 vs vehicle controls (vehicle).

FIG. 1B. Effect of sublingually administered Dexmedetomidine hydrochloride (Dex) at varying doses (0.5-3 μg/kg) on frequency of aggressive and agitated behaviors. Data expressed as Mean±SEM One-way ANOVA followed by Dunnett's post-hoc test *p<0.05 ** p<0.01, ***p<0.001 and ****p<0.0001 vs vehicle controls (vehicle).

FIG. 1C. Effect of intravenously administered Dexmedetomidine hydrochloride (Dex) at varying doses (0.5-3 μg/kg) on cumulative duration of aggressive and agitated behaviors. Data expressed as Mean±SEM. One-way ANOVA followed by Dunnett's post-hoc test. **p<0.05 **p<0.01, ***p<0.001 and ****p<0.0001 vs vehicle controls (vehicle).

FIG. 1D. Effect of intravenously administered Dexmedetomidine hydrochloride (Dex) at varying doses (0.5-3 μg/kg) on frequency of aggressive and agitated behaviors. Data expressed as Mean±SEM. One-way ANOVA followed by Dunnett's post-hoc test. *p<0.05 **p<0.01, ***p<0.001 and ****p<0.0001 vs vehicle controls (vehicle).

FIG. 2A. Effect of sublingually administered Dexmedetomidine hydrochloride (Dex) at varying doses (0.5-3 μg/kg) on Latency to attack. Data is expressed as Mean±SEM. Statistical analysis was performed by One-way ANOVA followed by Dunnett's post-hoc test. *p<0.05 **p<0.01, ***p<0.001 and ****p<0.0001 vs vehicle controls (vehicle).

FIG. 2B. Effect of intravenously administered Dexmedetomidine hydrochloride (Dex) at varying doses (0.5-3 μg/kg) on Latency to attack. Data is expressed as Mean±SEM Statistical analysis was performed by One-way ANOVA followed by Dunnett's post-hoc test. *p<0.05 **p<0.01, ***p<0.001 and ****p<0.0001 vs vehicle controls (vehicle).

FIG. 3A. Effect of sublingually administered Dexmedetomidine hydrochloride (Dex) at varying doses (0.5-3 μg/kg) on Cumulative duration of Neutral behaviors such as grooming, and exploration. Data expressed as Mean±SEM. Data is expressed as Mean±SEM. Statistical analysis was performed by One-way ANOVA followed by Dunnett's post-hoc test. *p<0.05 **p<0.01, ***p<0.001 and ****p<0.0001 vs vehicle controls (vehicle).

FIG. 3B. Effect of sublingually administered Dexmedetomidine hydrochloride (Dex) at varying doses (0.5-3 μg/kg) on Frequency of Neutral behaviors such as grooming, and exploration. Data expressed as Mean±SEM. Data is expressed as Mean±SEM. Statistical analysis was performed by One-way ANOVA followed by Dunnett's post-hoc test. *p<0.05 **p<0.01, ***p<0.001 and ****p<0.0001 vs vehicle controls (vehicle).

FIG. 3C. Effect of sublingually administered Dexmedetomidine hydrochloride (Dex) at varying doses (0.5-3 μg/kg) on Neutral behaviors such as immobile/quiet time. Data expressed as Mean±SEM. Data is expressed as Mean±SEM. Statistical analysis was performed by One-way ANOVA followed by Dunnett's post-hoc test. *p<0.05 **p<0.01, ***p<0.001 and ****p<0.0001 vs vehicle controls (vehicle).

FIG. 3D. Effect of intravenously administered Dexmedetomidine hydrochloride (Dex) at varying doses (0.5-3 μg/kg) on Cumulative duration of Neutral behaviors such as grooming, and exploration. Data expressed as Mean±SEM. Data is expressed as Mean±SEM. Statistical analysis was performed by One-way ANOVA followed by Dunnett's post-hoc test. *p<0.05 **p<0.01, ***p<0.001 and ****p<0.0001 vs vehicle controls (vehicle).

FIG. 3E. Effect of intravenously administered Dexmedetomidine hydrochloride (Dex) at varying doses (0.5-3 μg/kg) on Frequency of Neutral behaviors such as grooming, and exploration. Data expressed as Mean±SEM. Data is expressed as Mean±SEM. Statistical analysis was performed by One-way ANOVA followed by Dunnett's post-hoc test. *p<0.05 **p<0.01, ***p<0.001 and ****p<0.0001 vs vehicle controls (vehicle).

FIG. 3F. Effect of intravenously administered Dexmedetomidine hydrochloride (Dex) at varying doses (0.5-3 μg/kg) on Neutral behaviors such as immobile/quiet time. Data expressed as Mean±SEM. Data is expressed as Mean±SEM. Statistical analysis was performed by One-way ANOVA followed by Dunnett's post-hoc test. *p<0.05 ** p<0.01, ***p<0.001 and ****p<0.0001 vs vehicle controls (vehicle).

FIG. 4A: Mean plasma concentrations following Sublingual (SL) Dexmedetomidine hydrochloride administration in rats. Data expressed as Mean±SD

FIG. 4B: Mean plasma concentrations following Intravenous (IV) Dexmedetomidine hydrochloride administration in rats. Data expressed as Mean±SD

### DETAILED DESCRIPTION OF THE INVENTION

### I. Abbreviations

The following abbreviations are used throughout this specification:

AD: Alzheimer's disease

AUC: Area under the curve

BZDs: Benzodiazepines

CNS: Central nervous system

CT/CAT scan: computed tomography scan

Cₘₐₓ: Maximum (or peak) serum concentration that a drug achieves in a specified compartment

EPS: Extrapyramidal side effects

FD & C: Federal Food, Drug, and Cosmetic

FTD: Fronto-temporal dementia

GABA: Gamma-aminoautyric Acid

5-HT: 5-Hydroxytryptamine

ICU: Intensive care unit

IPD: In-Patient department

MRI: Magnetic resonance imaging

Mg: Milligram

NE: Nor-epinephrine

OPD: Out-patient department

PTSD: Post-traumatic stress disorders

RSS: Ramsay sedation score

RIT: Rat intruder test

SLOS: Smith-Lemli Opitz syndrome

Tₘₐₓ: Time at which the Cₘₐₓ is observed.

### II. Definitions

It will be understood that the terminology used herein is for the purpose of describing embodiments only, and is not intended to be limiting. As used in this specification, the singular forms “a”, “an” and “the” include plural referents unless the context clearly dictates otherwise. Thus, for example, reference to “a solvent” includes one or more such solvents and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

The terms “treating,” and “treatment,” as used herein refer to curative therapy, prophylactic therapy, and/or preventative therapy and can be used interchangeably.

As used herein, unless indicated otherwise, the terms “pharmaceutical composition”, “composition”, “formulation” and “composition of the invention,” are used interchangeably. Unless stated otherwise, the terms are meant to encompass, and are not limited to, pharmaceutical compositions containing drug substance i.e. Dexmedetomidine. The composition may also contain one or more “excipients” that are “inactive ingredients” or “compounds” devoid of pharmacological activity or other direct effect in the diagnosis, cure, mitigation, treatment, or prevention of disease or to affect the structure or any function of the human body.

As used herein, the term “an effective amount” is interchangeable with “therapeutically effective dose,” or “therapeutically effective amount,” and refers to an amount sufficient to produce the desired effect. An effective amount is sufficient to cause an improvement in a clinically significant condition of the subject.

As used herein, “pharmaceutically acceptable salt” refers to a salt known to be non-toxic and commonly used in the pharmaceutical literature. Typical inorganic acids used to form such salt include hydrochloric, hydrobromic, hydroiodic, nitric, sulfuric, phosphoric, hypophosphoric, and the like. Salts derived from organic acids, such as aliphatic mono and dicarboxylic acids, phenyl substituted alkanoic acids, hydroxyalkanoic and hydroxyl alkandioic acids, aromatic acids, aliphatic and aromatic sulfonic acids may also be used. A preferred salt is the hydrochloride salt.

As used herein, the term “subject” preferably refers to a human patient. In some embodiments, the subject can be any animal, including non-human mammals, such as mice, rats, other rodents, rabbits, dogs, cats, swine, cattle, sheep, horses, or primates.

The term “agitation”, as used herein, means irritability, emotional outburst, impaired thinking, or excess motor and verbal activity that may occur due to either dysfunction of specific brain regions such as frontal lobes or due to dysfunction of neurotransmitter systems such as dopamine and nor-epinephrine. In the present invention, agitation also includes aggression and hyper-arousal in post-traumatic stress disorder. The agitation may be acute or chronic.

The term “the signs of agitation” includes excessive motor activity (examples include: pacing, rocking, gesturing, pointing fingers, restlessness, performing repetitious mannerisms), verbal aggression (e g. yelling, speaking in an excessively loud voice, using profanity, screaming, shouting, threatening other people), physical aggression (e.g.. grabbing, shoving, pushing, clenching hands into fists, resisting, hitting others, kicking objects or people, scratching, biting, throwing objects, hitting self, slamming doors, tearing things, and destroying property).

The term “acute agitation” means agitation that occurs rapidly and is severe and sudden in onset. Acute agitation may be associated with, for example, neurodegenerative disease and neuropsychiatric conditions, although it may particularly exist in neuropsychiatric conditions. Acute agitation may lead to chronic agitation if it remains untreated.

The term “chronic agitation” means agitation developed over a long period of time, and is less severe than acute agitation. Chronic agitation may be associated with, for example, neurodegenerative disease and neuropsychiatric conditions, although it may particularly exist in neurodegenerative diseases.

The term “neurodegenerative disease” includes, but is not limited to, Alzheimer disease, frontotemporal dementia (or Pick's disease), Dementia, Dementia with Lewy bodies, post-traumatic stress disorder, Parkinson's disease, vascular dementia, vascular cognitive impairment, Huntington's disease, multiple sclerosis, Creutzfeldt-Jakob disease, multiple system atrophy, progressive supranuclear palsy or other related neurodegenerative diseases.

The term “neuropsychiatric conditions” includes, but is not limited to, schizophrenia, bipolar illness (bipolar disorder, bipolar mania), depression, delirium or other related neuropsychiatric conditions.

“Sundown syndrome” is a late-day circadian syndrome of increased confusion and restlessness, generally in a patient with some form of dementia. It seems to occur more frequently during the middle stages of Alzheimer dementia. It seems to subside with the progression of a patient's dementia. About 20-45% of Alzheimer type patients will experience some sort of sundowning confusion. Confusion and agitation worsen in the late afternoon and evening, or as the sun goes down.

The term “perioperative agitation” means agitation before, during or after any surgical procedure or ICU agitation unassociated with a neurodegenerative disease or neuropsychiatric condition.

The term “sublingual” literally means “under the tongue” and refers to a method of administering substances via the mouth in such a way that the substances are rapidly absorbed via the blood vessels under the tongue rather than via the digestive tract. Sublingual absorption occurs through the highly vascularized sublingual mucosa, which allows a substance direct access to the blood circulation, thereby providing for direct systemic administration independent of gastrointestinal influences and avoiding undesirable first-pass hepatic metabolism. Accordingly, the total amount of Dexmedetomidine or a pharmaceutically acceptable salt thereof in the formulation may be reduced, thereby reducing the likelihood of deleterious side effects and providing a cost benefit to the manufacturer.

“Sedation” as used herein means depressed consciousness in which a patient or subject retains the ability to independently and continuously maintain an open airway and a regular breathing pattern, and to respond appropriately and rationally to physical stimulation and verbal commands. As used herein “without causing significant sedation” means that the patient experiences a level of sedation not greater than Level 3 on the Ramsay Sedation Scale. Level 3 means sedated but responds to commands.

### III. Methods

The present invention provides a method of treating agitation or the signs of agitation in a subject comprising administering an effective amount of an alpha-2 adrenergic agonist or a pharmaceutically acceptable salt thereof sublingually to the subject. In a particular aspect, the agitation is suppressed without also causing significant sedation.

In one embodiment, the alpha-2 adrenergic agonist includes, but is not limited to, Clonidine, Guanfacine, Guanabenz, Guanoxabenz, Guanethidine, Xylazine, Tizanidine, Medetomidine, Dexmedetomidine, Methyldopa, Methylnorepinephrine, Fadolmidine, Iodoclonidine, Apraclonidine, Detomidine, Lofexidine, Amitraz, Mivazerol, Azepexol, Talipexol, Rilmenidine, Naphazoline, Oxymetazoline, Xylometazoline, Tetrahydrozoline, Tramazoline, Talipexole, Romifidine, propylhexedrine, Norfenefrine, Octopamine, Moxonidine, Lidamidine, Tolonidine, UK14304, DJ-7141, ST-91, RWJ-S2353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof.

In one preferred embodiment, the present invention provides a method of treating agitation or the signs of agitation in a subject comprising administering an effective amount of Dexmedetomidine or a pharmaceutically acceptable salt thereof sublingually to the subject. In a particular aspect, the agitation is suppressed without also causing significant sedation.

Agitation may be effectively treated using a relatively low dose of Dexmedetomidine or a pharmaceutically acceptable salt thereof via the sublingual route. Consequently, in addition to providing relief from agitation without causing significant sedation, the treatment is also effective with reduced or no side effects (for example, cardiac or respiratory side effects).

In a further embodiment, the present invention is directed to a method of treating agitation or the signs of agitation in a subject comprising administering Dexmedetomidine or a pharmaceutically acceptable salt thereof sublingually to the subject to provide fast-acting relief without a substantial portion of Dexmedetomidine or its pharmaceutically acceptable salt thereof passing into the liver of the patient.

In another embodiment, the present invention provides a method of treating agitation or the signs of agitation in a subject in need thereof, comprising administering an effective amount of Dexmedetomidine or a pharmaceutically acceptable salt thereof via a sublingual composition to the subject, wherein the sublingual composition is selected from a film, wafer, patch, lozenge, gel, spray, tablet, and liquid drops.

In a further embodiment, the present invention provides a method of treating agitation or the signs of agitation in a subject in need thereof, comprising administering to the subject an effective amount of an alpha-2 adrenergic agonist together with one or more pharmaceutically acceptable carriers and/or excipients via a sublingual composition, wherein the sublingual composition is a sublingual film. In a particular aspect, the agitation is associated with a neurodegenerative disease or neuropsychiatric condition. In another particular aspect, the treatment is effective without causing significant sedation.

In a further embodiment, the present invention provides a method of treating agitation or the signs of agitation in a subject in need thereof, comprising administering to the subject an effective amount of Dexmedetomidine or a pharmaceutically acceptable salt thereof together with one or more pharmaceutically acceptable carriers and/or excipients via a sublingual composition, wherein the sublingual composition is a sublingual film. In a particular aspect, the agitation is associated with a neurodegenerative disease or neuropsychiatric condition. In another particular aspect, the treatment is effective without causing significant sedation.

In yet other embodiment, the present invention provides a method of treating agitation or signs of agitation in a subject in need thereof, comprising administering to said subject an effective amount of an alpha-2 adrenergic agonist or a pharmaceutically acceptable salt thereof at a dosage that does not cause a significant sedation. Suitable alpha-2 adrenergic agonists include, but are not limited to, Clonidine, Guanfacine, Guanabenz, Guanoxabenz, Guanethidine, Xylazine, Tizanidine, Medetomidine, Dexmedetomidine, Methyldopa, Methylnorepinephrine, Fadolmidine, Iodoclonidine, Apraclonidine, Detomidine, Lofexidine, Amitraz, Mivazerol, Azepexol, Talipexol,

Rilmenidine, Naphazoline, Oxymetazoline, Xylometazoline, Tetrahydrozoline, Tramazoline, Talipexole, Romifidine, propylhexedrine, Norfenefrine, Octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof. In a particular aspect of the invention, the dosage of alpha-2 adrenergic agonist used in the composition is from about 3 micrograms to about 100 micrograms.

In another embodiment, the present invention provides a method of treating agitation or the signs of agitation in a subject in need thereof, comprising administering to said subject an effective amount of Dexmedetomidine or a pharmaceutically acceptable salt thereof sublingually at a dosage that does not cause significant sedation. In a particular aspect of the invention, the dosage of Dexmedetomidine or a pharmaceutically acceptable salt thereof used in the sublingual composition is from about 3 micrograms to about 100 micrograms. Examples of suitable dosages include: about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms (e.g.. about 5, 6, 7, 8, or 9 micrograms), about 10 micrograms, about 12 micrograms, about 14 micrograms, about 15 micrograms, about 16 micrograms, about 18 micrograms, about 20 micrograms, about 30 micrograms, about 50 micrograms. The dose may be administered one or more times a day.

In a further embodiment, the present invention provides a method of treating agitation or the signs of agitation in a subject in need thereof, comprising administering to said subject an effective amount of Dexmedetomidine or a pharmaceutically acceptable salt thereof sublingually at a dosage of from about 0.05 micrograms/kg weight of subject to about 1.5 micrograms/kg weight of subject. Examples of suitable dosages include: about 0.1 micrograms/kg to about 1 micrograms/kg, about 0.1 micrograms/kg to about 0.5 micrograms/kg, about 0.1 micrograms/kg to about 0.4 micrograms/kg, about 0.1 micrograms/kg to about 0.3 micrograms/kg, about 0.1 micrograms/kg to about 0.2 micrograms/kg, about 0.07 micrograms/kg, about 0.05 micrograms/kg, about 0.1 micrograms/kg, about 0.2 micrograms/kg, about 0.3 micrograms/kg, about 0.4 micrograms/kg, about 0.5 micrograms/kg, about 0.6 micrograms/kg, about 0.7 micrograms/kg, about 0.8 micrograms/kg, about 0.9 micrograms/kg, about 1.0 micrograms/kg, about 1.1 micrograms/kg, about 1.2 micrograms/kg, about 1.3 micrograms/kg, about 1.4 micrograms/kg, about 1.5 micrograms/kg. The dose may be administered one or more times a day.

In yet other embodiment, the present invention provides a method of treating agitation or signs of agitation associated with neurodegenerative disease in a subject in need thereof, comprising administering to said subject an effective amount of an alpha-2 adrenergic agonist or a pharmaceutically acceptable salt thereof at a dosage that does not cause a significant sedation. Suitable alpha-2 adrenergic agonists include, but are not limited to, Clonidine, Guanfacine, Guanabenz, Guanoxabenz, Guanethidine, Xylazine, Tizanidine, Medetomidine, Dexmedetomidine, Methyldopa, Methylnorepinephrine, Fadolmidine Iodoclonidine, Apraclonidine, Detomidine, Lofexidine, Amitraz, Mivazerol, Azepexol, Talipexol, Rilmenidine, Naphazoline, Oxymetazoline, Xylometazoline, Tetrahydrozoline, Tramazoline, Talipexole, Romifidine, propylhexedrine, Norfenefrine, Octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof. The dosage of alpha-2 adrenergic agonist used in the composition is conveniently from about 3 micrograms to about 100 micrograms.

In a yet further embodiment, the present invention provides a method of treating agitation or the signs of agitation associated with neurodegenerative disease in a subject in need thereof, comprising sublingually administering to said subject an effective amount of Dexmedetomidine or a pharmaceutically acceptable salt thereof at a dosage that does not cause unwanted (e.g.., significant) sedation. The dosage of Dexmedetomidine or a pharmaceutically acceptable salt thereof used may conveniently be from about 3 micrograms to about 100 micrograms, e.g.. about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms, about 5 micrograms, about 6 micrograms, about 7 micrograms, about 8 micrograms, about 9 micrograms, about 10 micrograms, about 12 micrograms, about 14 micrograms, about 16 micrograms, about 18 micrograms. The dose may be administered one or more times a day.

In yet other embodiment, the present invention provides a method of treating agitation or signs of agitation associated with neuropsychiatric condition in a subject in need thereof, comprising administering to said subject an effective amount of an alpha-2 adrenergic agonist or a pharmaceutically acceptable salt thereof at a dosage that does not cause a significant sedation. Suitable alpha-2 adrenergic agonists include, but are not limited to, Clonidine, Guanfacine, Guanabenz, Guanoxabenz, Guanethidine, Xylazine, Tizanidine, Medetomidine, Dexmedetomidine, Methyldopa, Methylnorepinephrine, Fadolmidine, Iodoclonidine, Apraclonidine, Detomidine, Lofexidine, Amitraz, Mivazerol, Azepexol, Talipexol, Rilmenidine, Naphazoline, Oxymetazoline, Xylometazoline, Tetrahydrozoline, Tramazoline, Talipexole, Romifidine, propylhexedrine, Norfenefrine, Octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof. The dosage of alpha-2 adrenergic agonist used in the composition is conveniently from about 3 micrograms to about 100 micrograms.

In another embodiment, the present invention provides a method of treating agitation or the signs of agitation associated with neuropsychiatric condition in a subject in need thereof, comprising administering to said subject an effective amount of Dexmedetomidine or a pharmaceutically acceptable salt thereof sublingually at a dosage that does not cause significant sedation. The dosage of Dexmedetomidine or a pharmaceutically acceptable salt thereof used in a sublingual composition may conveniently be from about 3 micrograms to about 100 micrograms, e.g.. about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms, about 5 micrograms, about 6 micrograms, about 7 micrograms, about 8 micrograms, about 9 micrograms, about 10 micrograms, about 12 micrograms, about 14 micrograms, about 15 micrograms, about 16 micrograms, about 18 micrograms, about 20 micrograms, about 30 micrograms, about 50 micrograms. The dose may be administered one or more times a day.

The level of acceptable sedation when treating a subject according to a method of the present invention is preferably at or below Level 3 according to the Ramsay sedation scoring (RSS) system. Thus, a particular embodiment of the present invention provides a method of treating agitation or the signs of agitation in a human subject in need thereof, comprising administering Dexmedetomidine or a pharmaceutically acceptable salt thereof sublingually to said subject at a dose in the range of about 3 micrograms to about 100 micrograms, thereby achieving an RSS at or below Level 3 (e.g.. Level 2 or Level 3).

### IV. Pharmaceutical Compositions

The present invention also provides sublingual pharmaceutical compositions comprising an effective amount of an alpha-2 adrenergic agonist or a pharmaceutically acceptable salt thereof, preferably Dexmedetomidine or a pharmaceutically acceptable salt thereof.

The sublingual pharmaceutical compositions of the present invention may also comprise a pharmaceutically acceptable carrier and/or excipient. Suitable pharmaceutically acceptable carriers include water, sodium chloride, binders, penetration enhancers, diluents, lubricants, flavouring agents, coloring agents and so on.

The sublingual pharmaceutical compositions of the present invention may be administered to a subject alone or in combination with one or more other suitable active ingredients.

In one embodiment, the present invention provides a sublingual pharmaceutical composition comprising an effective amount of Dexmedetomidine or a pharmaceutically acceptable salt thereof for the treatment of agitation in a subject, e.g.., agitation associated with neurodegenerative disease, sundown syndrome in Alzheimer's disease or dementia. In a particular aspect, the sublingual pharmaceutical composition effectively treats agitation in a subject without causing significant sedation.

In another embodiment, the present invention provides a sublingual pharmaceutical composition comprising an effective amount of Dexmedetomidine or a pharmaceutically acceptable salt thereof for the treatment of agitation in a subject associated with schizophrenia, bipolar disorder, bipolar mania, other bipolar illness, depression, delirium or another related neuropsychiatric condition. In a particular aspect, the sublingual pharmaceutical composition effectively treats agitation in a subject without causing significant sedation.

The sublingual pharmaceutical composition of the present invention may be, for example, a film, wafer, patch, lozenge, gel, spray, tablet, liquid drops or the like.

In one embodiment of the invention, the sublingual pharmaceutical composition is in the form of a tablet or packed powder.

In another embodiment of the invention, the sublingual pharmaceutical composition is in the form of a patch or film (e.g.. thin film). The patch may have adhesive qualities to prevent movement or swallowing of the patch. The patch may be ingestible in case of accidental swallowing or to allow for its easy disposal, or the patch may be removed from under the tongue after a prescribed time.

In yet another embodiment of the invention, the sublingual pharmaceutical composition is in the form of a paste, gel or ointment. The viscosity of the paste, gel or ointment can be adjusted to allow for retention under the tongue.

In a further embodiment of the invention, the sublingual pharmaceutical composition is in a liquid (e.g.. as a solution, suspension or emulsion), and may be, for example, presented as a spray or as drops. Solutions include the active ingredient together with a diluent such as water, normal saline, sodium chloride solution, or any other suitable solvent such as propylene glycol, glycerol, ethyl alcohol and so on. The diluent for the solution may particularly be physiological saline solution or water. The amount of solution administered may conveniently be about 0.01 ml to about 1 ml (e.g.., about 0.025-0.5 ml).

The non-solid compositions of the invention may conveniently be administered by spraying, dripping, painting or squirting the composition under the tongue.

In a particular embodiment of the invention, Dexmedetomidine or a pharmaceutically acceptable salt thereof is sublingually administered in liquid form, e.g.. in a flavored or unflavored physiological saline solution. The liquid composition may conveniently be administered under the tongue as drops or as a spray.

Dexmedetomidine, or a pharmaceutically acceptable salt thereof may conveniently represent from about 0.001% to about 99.99% of the overall composition, e.g.. about 0.01% to about 90%, more particularly about 0.01% to about 30%.

When the composition is a liquid or gel, a first unit dose is applied and held in place under the tongue for a predetermined time, for example for at least about 30 seconds, or more particularly about 60 seconds or more. A second unit dose may then be applied and held in place for a similar amount of time. Surprisingly, this procedure noticeably increases the effect of the composition of the invention in the treatment of agitation or the signs of agitation.

In another embodiment, the sublingual composition of Dexmedetomidine or a pharmaceutically acceptable salt thereof is a hard tablet or a compressed powder tablet. The tablet may conveniently be designed to dissolve under the tongue in about 30 to 120 seconds as disclosed in U.S. Pat. No. 6,221,392 to Khankari, et al., incorporated herein by reference. In a particular embodiment, the sublingual composition of Dexmedetomidine or a pharmaceutically acceptable salt thereof is a hard tablet having a low grit component for an organoleptically pleasant mouth feel. The tablet (or particles thereof containing the active ingredient which can be compressed to form the tablet) may also comprise a protective outer coating, e.g.. any polymer conventionally used in the formation of microparticles, matrix-type microparticles and microcapsules.

In a further embodiment, the sublingual composition of Dexmedetomidine or a pharmaceutically acceptable salt thereof is a hard, compressed, rapidly dissolvable tablet The tablet conveniently includes the active ingredient within a matrix. The matrix may be composed of, for example, at least one filler and a lubricant. Fillers include, for example, lactose or mannitol, and suitable lubricants include magnesium stearate, silicon dioxide and talc. The matrix may also include one or more of: a binder (e.g.. povidone, a sugar or carboxymethylcellulose), a disintegrant (e.g.. croscarmellose sodium, crospovidone or sodium starch glycolate), a sweeting agent (e.g.. sucralose) and the like. The tablet may conveniently have a friability of about 2% or less and a hardness of about 15 to about 50 Newtons.

Another aspect of the present invention provides a method of making a packaged, sublingual tablet. The method includes the steps of: (a) forming a mixture comprising Dexmedetomidine or a pharmaceutically acceptable salt thereof and a matrix including at least a non-direct compression filler and a lubricant: (b) compressing the mixture to form a plurality of hard, compressed, rapidly disintegrable particles (e.g.. beads) including the active ingredient distributed in the sublingually dissolvable matrix; and (c) storing the product in bulk prior to packaging In another embodiment, the dosage forms are then packaged in a lumen of a package such that there are more than one per package. Direct compression is the preferred method of forming the dosage forms. There is also provided hereby an openable and reclosable package containing a plurality of hard, compressed, rapidly dissolving tablets adapted for direct oral dosing as described above.

In another embodiment, the present invention is a sublingual tablet comprising an effervescent agent. The effervescent agent may conveniently be present in an amount up to about 95% by weight, based on the weight of the finished tablet, and more particularly in an amount of between about 30% and about 80% by weight. Sufficient effervescent material is included in the tablet composition to generate more than about 5 cm³ but less that about 30 cm³ of gas upon exposure of the tablet to an aqueous environment. Sublingual compositions comprising effervescent agents are disclosed in U.S. Pat. No. 6,200,604, which is incorporated herein by reference.

In one particular embodiment, an effervescent agent releases carbon dioxide e.g.., as a result of the reaction of a soluble acid source with an alkaline carbonate or bicarbonate. The acid source may conveniently include food acids and acids such as citric acid, tartaric, amalic, fumeric, adipic and succinic acid. Carbonate and bicarbonate sources include dry solid carbonate and bicarbonate salts such as sodium bicarbonate, sodium carbonate, potassium bicarbonate, potassium carbonate, magnesium carbonate and the like.

Spray compositions of the present invention for sublingual administration may include one or more pharmaceutically acceptable liquids (e.g.., present in the amount of about 30% to about 99.99% by weight of the composition). Such liquids may be solvents, co-solvents, or non-solvents for Dexmedetomidine or a pharmaceutically acceptable salt thereof. Examples of pharmaceutically acceptable liquids include water, ethanol, dimethyl sulfoxide, propylene glycol, polyethylene glycol, propylene carbonate, pharmaceutically acceptable oils (e.g.., soybean, sunflower, peanut, peppermint etc.) and the like. The pharmaceutically acceptable liquid is selected either to dissolve the active pharmaceutical ingredient, to produce a stable, homogenous suspension or solution of it, or to form any combination of a suspension or solution.

Furthermore, sublingual, spray formulations of Dexmedetomidine or a pharmaceutically acceptable salt thereof may include one or more carriers and/or excipients Examples of carriers/excipients include viscosity-modulating materials (e.g.. polymers, sugars, sugar alcohols, gums, clays, silicas, and the like). One particular polymer that may conveniently be used is polyvinylpyrrolidone (PVP). The viscosity-modulating material may conveniently be present in the amount of from about 0.01% to about 65% by weight of the spray formulation. Other examples of carriers/excipients include preservatives (e.g.. ethanol, benzyl alcohol, propylparaben and methylparaben). Preservatives may conveniently be present in the amount of from about 0.001% to about 10% by weight of the spray formulation. Carriers/excipients may also be flavoring agents, sweeteners (e.g.. sugars such as sucrose, glucose, dextrose, maltose, fructose, etc.), artificial sweeteners (e.g. saccharin, aspartame, acesulfame, sucralose etc), or sugar alcohols (e.g.. mannitol, xylitol, lactitol, maltitol syrup etc.) present conveniently in an amount of from about 0.001% to about 65% by weight of the spray formulation. Other examples of carriers/excipients include buffers and pH-adjusting agent (e.g.., sodium hydroxide, citrate, and citric acid) conveniently present in an amount of from about 0.01% to about 5% by weight of the spray formulation. Coloring agents (e.g.. present in an amount of from about 0.001% to about 5% by weight of the spray formulation), fragrances (e.g.. present in an amount of from about 0.001% to about 1% by weight of the spray formulation), chelating agents such as EDTA (e.g.. present in an amount of from about 0.001% to about 1% by weight of the spray formulation), UV absorbers (e.g.. present in an amount of from about 0.001% to about 10% by weight of the spray formulation), and anti-foam agents (e.g.. low molecular weight alcohols, dimethicone) conveniently present in an amount of from about 0.001% to about 5% by weight of the spray formulation may also be included as appropriate carriers/excipients in the spray formulations of the present invention.

One particular aspect of the present invention provides a sublingual film comprising Dexmedetomidine or a pharmaceutically acceptable salt thereof, together with one or more carriers and/or excipients, for the treatment of agitation.

Excipients which may be incorporated into the sublingual films of the present invention include one or more of the following: film forming agents, mouth feel improvers, plasticizers, stabilizers, surfactants, preservatives, sweetening agents, colorants, flavourants, emulsifiers, disintegrants, salivating agents, antioxidants, permeation enhancers, solvents and the like.

Film forming agents generally mean agents that provide structure to the film of the present invention. The effective amount of the film forming agent ranges from about 10% to about 99%, more preferably about 50% to about 90% by weight of the composition. Film forming agents that can be utilized as part of the film composition of the present invention include, but are not limited to, cellulose ethers, modified starches, natural gums, edible polymers, seaweed extracts, land plant extracts, pullulan, polyvinylpyrrolidone, derivatives thereof and combinations thereof.

Examples of cellulose ethers include, but are not limited to, methylhydroxycellulose, methylcellulose, ethylcellulose, hydroxyethylcellulose, carboxymethylcellulose, derivatives thereof and combinations thereof.

Modified starches include, but are not limited to, acid and enzyme hydrolyzed corn and potato starches, derivatives thereof and combinations thereof.

Examples of natural gums include, but are not limited to, gum arabic, guar gum, locust bean gum, carrageenan gum, acacia, karaya, ghatti, tragacanth agar, tamarind gum, xanthan gum, derivatives thereof and combinations thereof.

Examples of edible polymers include, but are not limited to, microcrystalline cellulose, cellulose ethers, xanthan, derivatives thereof and combinations thereof.

Seaweed extract examples include, but are not limited to, sodium alginate, carrageenans, derivatives thereof and. combinations thereof.

Land plant extracts include, but are not limited to, konjac, pectin, arabinoglactan, derivatives thereof and combinations thereof.

Particular film forming agents include pullulan, sodium alginate, polyvinylpyrrolidone, methylcellulose and methylhydroxycellulose (MHC).

The term “solvent” generally refers to liquids that will dissolve solutes. A solvent may be used to dissolve film-forming agents and other excipients to prepare film-forming compositions of the present invention. Solvents include, but are not limited to, demineralized/distilled water, ethyl alcohol, isopropyl alcohol, methyl ethyl ketone, propylene glycol methyl ether acetate, dimethyl acetamide, ethylene glycol mono-propyl ether, and toluene. A sublingual film of the present invention may conveniently comprise a solvent in an amount up to about 1% w/w.

The term “stabilizer” generally refers to an agent that will impart stability to the formulation during its shelf life. Stabilizers of the present invention can include, for example, oil/water emulsifiers and flavor fixatives. The effective amount of a stabilizer agent in a composition of the invention may be, for example, in the range of about 0% to about 45%, more particularly about 4% to about 25%, by weight of the composition. Examples of suitable stabilizing agents of the present invention include, but are not limited to, gum arabic, microcrystalline cellulose, carrageenan, xanthan gum, locust bean gum, derivatives thereof and combinations thereof. Particular stabilizing agents of the present invention include gum arabic and microcrystalline cellulose.

“Disintegrants” can aid the dissolution of edible films allowing for the efficacy of the film to be realized sooner. Suitable disintegrants for use in an edible film of the present invention include, but are not limited to, alginic acid, microcrystalline cellulose and carboxymethylcellulose. Special disintegrants known as super-disintegrants are also suitable for use in an edible film of the present invention. Super-disintegrants include cross-linked polymers (e g. crospovidone), cross-linked starches (e.g.. sodium starch glycolate), and cross-linked celluloses (e.g.. a modified carboxymethylcellulose such as croscarmellose). These super-disintegrants are insoluble in water and most other solvents, have rapid swelling properties, and have good water uptake with high capillary action, resulting in fast disintegration. Their insolubility in many solvents also means they enable the manufacture of sublingual compositions of this invention in a single step process as opposed to costly multi-step processes.

The disintegrants or super-disintegrants are conveniently present in a sublingual composition of this invention (e.g.. an edible film) in an amount ranging from about 1% to about 10%, more particularly about 1% to about 5% by weight of the composition.

“Emulsifiers” suitable for use in an edible film of the present invention include, but are not limited to, gum arabic, carrageenan, triethanolamine stearate, quaternary ammonium compounds, acacia, gelatin, lecithin, bentonite, veegum, derivatives thereof and combinations thereof. Emulsifiers can be used in a composition of the present invention in an amount up to about 40%, more particularly up to about 25%, by weight of the composition. The emulsifier can be a stabilizer creating an oil/water emulsion encapsulating volatile oils and flavoring agents, thereby essentially acting as a flavor fixative. A particular emulsifier for use in an edible film of the present invention is gum arabic.

A “plasticizing agent” or “plasticizer” may be utilized to improve flexibility and reduce brittleness of an edible film composition of the present invention. The plasticizing agent may conveniently constitute up to about 30%, e.g.. up to about 15% by weight of the composition. Examples of suitable plasticizing agents include, but are not limited to, glycerin, sorbitol, triacetin, monoacetin, diacetin, polyethylene glycol, propylene glycol, hydrogenated starch hydrolysates, corn syrups, low molecular weight propylene glycols, phthalate derivatives like dimethyl, diethyl and dibutyl phthalate, citrate derivatives such as tributyl, trietbyl, acetyl citrate and castor oil derivatives thereof and combinations thereof. Particular plasticizing agents of the present invention include sorbitol and glycerin.

The term “preservative” generally refers to an excipient used to kill microorganisms or prevents, inhibits or retards their growth and reproduction, and is included in a product in a concentration only sufficient to prevent spoilage or the growth of inadvertently added microorganisms. Suitable preservative includes, but are not limited to, methylparaben, propylparaben and sodium benzoate. The preservative may conveniently be present in the composition from about 0.001% to about 10% w/w of the composition.

The term “sweetening agent” generally refers to an excipient used to impart sweetness to a pharmaceutical composition. Suitable sweetening agents for use in a composition of the present invention include, but are not limited to, aspartame, dextrose, glycerin, mannitol, saccharin sodium, sorbitol and sucrose. The sweetening agent may conveniently be present in the composition in an amount of from about 5% to about 20% w/w of the composition.

The term “coloring agent” or “colorant” generally refers to an excipient used to impart color to a pharmaceutical composition. Suitable colorants include, but are not limited to, FD&C Red No 3, FD&C Red No. 20, FD&C Yellow No. 6, FD&C Blue No. 2, D&C Green No. 5, D&C Orange No. 5, D&C Red No. 8, other F.D. & C. dyes, caramel, red ferric oxide, and natural coloring agents such as grape skin extract, beet red powder, beta-carotene, annatto, carmine, turmeric or paprika. The colorant may conveniently be present in the composition in an amount of from about 0.001% to about 10% w/w of the composition.

The term “flavoring agent” or “flavorant” generally refers to an excipient used to impart a pleasant flavor (and often also odor) to a pharmaceutical composition. Suitable flavorants include, but are not limited to, synthetic flavoring oils, flavoring aromatics, natural oils, extracts from whole plants or parts thereof such as leaves, flowers, fruits or combinations thereof. Examples include cinnamon oil, wintergreen oil, peppermint oil, clove oil, bay oil, anise oil, eucalyptus oil, thyme oil, cedar leave oil, nutmeg oil, sage oil, bitter almond oil and cassia oil. Other useful flavorants include vanilla, citrus fruit oils such as lemon, orange, grape, lime or grapefruit oil, and fruit essences such as apple, pear, peach, strawberry, raspberry, cherry, plum, pineapple or apricot essence. Flavorants of particular interest for use in a composition of the present invention include commercially available orange, grape, cherry and bubble gum flavors and mixtures thereof. The amount of flavoring used will depend on a number of factors, including the organoleptic effect desired. Particular flavorants include grape and cherry flavors, and citrus fruit flavors such as orange flavor. The flavorant may conveniently be present in the composition in an amount of from about 0.001% to about 10% w/w of the composition.

The term “salivating agent” is an agent that promotes greater salivation during use of a composition of the present invention. This may be an important feature if the composition is intended to be taken by the patient without the aid of water to help in the transporting of the composition to the stomach of the patient. The salivating agent can be, for example, an emulsifier or a food acid that initiates salivation in the mouth of the patient. Examples of emulsifiers useful as salivating agents include alkyl aryl sulfonates, alkyl sulfates, sulfonated amides and amines, sulfated and sulfonated esters and ethers, alkyl sulfonates, polyethoxylated esters, mono-, di-, and triglycerides, diacetyl tartaric esters of monoglycerides, polyglycerol esters, sorbitan esters and ethoxylates, lactylated esters, phospholipids such as lecithin, polyoxyethylene sorbitan esters, proplyene glycol esters, sucrose esters, and mixtures thereof. The emulsifier may be either saturated or unsaturated. It should be noted that some of the emulsifiers that are salivating agents may also function as binders. Examples of food acids useful as salivating agents include citric acid, malic acid, tartarate, food salts such as sodium chloride and salt substitutes, potassium chloride, and mixtures thereof. The amount of salivating agent present in a sublingual film of the present invention may convenient be up to about 15% by weight of the final composition, e.g.. in the range of from about 0.3% to 0.4% by weight of the composition.

The term “antioxidant” generally refers to an excipient used to inhibit oxidation and thus prevent deterioration of active agents by oxidative processes. Suitable antioxidants include, for example, ascorbic acid, ascorbyl palmitate, butylated hydroxyanisole, butylated hydroxytoluene, hypophosphorous acid, monothio-glycerol, propyl gallate, sodium ascorbate, citric acid, sodium bisulfite, sodium formaldehyde sulfoxylate, sodium metabisulfite, EDTA and sodium edetate. The anti-oxidant may conveniently be present in the composition in an amount of from about 0.001% to about 2% w/w of the composition.

The term “permeation enhancer” generally refers to an excipient used to enhance permeation of an active agent to cellular membranes or enhance the local/systemic absorption of the active agent. Permeation enhancers that may be used in the present invention include, but are not limited to, solubilizers such as alcohols, polyethylene glycols, chelating agents (e.g.. cyclodextrins), sucrose laurate or sucrose oleate. The permeation enhancer may conveniently be present in the composition in an amount of from about 0.1% to about 5% w/w of the composition.

In one embodiment of the present invention, the sublingual pharmaceutical composition of the present invention includes a mucosal permeation enhancer appropriate for enhancing the mucosal absorption of the composition.

Sublingual Dexmedetomidine formulations (such as sprays, drops, and the like) may be made by mixing appropriate quantities of the foregoing ingredients in accordance with standard good manufacturing practices. The relative amounts of each ingredient should not interfere with the desirable pharmacological and pharmacokinetic properties of the resulting formulation.

Sublingual Dexmedetomidine films of the present invention may be conveniently prepared using PharmFilm® technology (owned by MonoSol) or technology owned by ARx LLC. Various patents and patent applications are incorporated herein in entirety and includes U.S. Pat. or Publication Nos. U.S. Pat. Nos. 9,585,961, 7,470,397, 7,727,466, 9,248,146, 9,545,376, 2017-0087084, U.S. Pat. Nos. 9,662,297, 9,662,301, 2017-0246108, 2017-0252294, U.S. Pat. No. 9,441,142 assigned to ARx LLC and U.S. Pat. Nos. 7,425,292, 7,357,891, 8,663,687, 8,685,437, 7,897,080, 8,241,661, 8,617,589, 8,936,825, 9,561,191, 9,303,918, 9,346,601, 8,282,954, 7,972,618, 9,073,294 assigned to Monosol Rx.

In preparing the sublingual film of the present invention the active agent, e.g.. Dexmedetomidine or a pharmaceutically acceptable salt thereof, film forming agents and optionally one or more carriers and/or excipients selected from the group comprising of mouth feel improver, plasticizer, stabilizer, surfactant, preservative, sweetening agent, colorant, flavourant, emulsifier, disintegrant, salivating agent, antioxidant, permeation enhancer are dissolved in a compatible solvent to form a film forming composition. Compatible solvents include water, alcohols such as ethanol, ethyl acetate, acetone, and mixtures thereof. The film forming composition is cast on a releasable carrier and dried to form a sheet/film. The carrier material must have a surface tension which allows the film solution to spread evenly across the intended carrier width without soaking to form a destructive bond between the film carrier substrates. Examples of suitable carrier materials include glass, stainless steel, Teflon and polyethylene-impregnated paper. Drying of the film may be carried out at high temperature using a drying oven, drying terminal, vacuum drier, or any other suitable drying equipment which does not adversely affect the ingredients of which the film is composed. The sublingual film of the present invention can also be prepared by other established processes e.g.. extrusion (for example, Hot melt extrusion, Solid dispersion extrusion), casting (for example, solid casting or semi-solid casting), Rolling methods and the like.

### V. Administration

In an aspect, the present invention provides a sublingual composition comprising an alpha-2 adrenergic agonist or a pharmaceutically acceptable salt thereof, administered to a subject in an amount sufficient to effectively treat agitation. The amount of alpha-2 adrenergic agonist is sufficient to effectively treat agitation without causing significant sedation The alpha-2 adrenergic agonist may conveniently be delivered on an “as needed basis” in one, two or more doses per day to the animal (e.g.. human) subject. The composition may also be administered via a single dosage form or via multiple dosage forms.

In another aspect, the present invention provides a sublingual composition comprising Dexmedetomidine or a pharmaceutically acceptable salt thereof, administered to a subject in an amount sufficient to effectively treat agitation. In a particular aspect, the amount of Dexmedetomidine or a pharmaceutically acceptable salt thereof used is sufficient to effectively treat agitation without causing significant sedation. The Dexmedetomidine or a pharmaceutically acceptable salt thereof may conveniently be delivered on an “as needed basis” in one, two or more doses per day to the animal (e.g.. human) subject. The composition may also be administered via a single dosage form or via multiple dosage forms.

Following administration of a composition of this invention to a subject, a therapeutic (i.e. anti-agitation) effect may begin within about 60 minutes (e.g.. within about 30, 20, 15, 10, 5, 3, 2 or 1 minutes) after administration, or within about 30 seconds after administration. The signs of agitation may also be relieved within about 1 to about 60 minutes after administration, and more typically within about 5 to about 30 minutes. A second dose of the composition of this invention may be administered to the subject if the signs of agitation are not relieved within about 60 minutes.

Treatment protocols may include one or more dosage intervals (e.g.. two or more dosage intervals, five or more dosage intervals, or ten or more dosage intervals). Depending on the physiology of the subject and the desired therapeutic effect, the duration of dosage intervals and treatment protocols according to embodiments of the present invention may vary.

Dexmedetomidine or a pharmaceutically acceptable salt thereof may be administered as a sublingual composition to treat agitation or the signs of agitation either alone or in combination with one or more further active agents. When used in combination, the active agents can either be formulated as a single composition or as two or more separate compositions, which can be administered simultaneously, sequentially or separated by an appropriate period of time.

Where Dexmedetomidine or a pharmaceutically acceptable salt thereof is administered with a second active agent to treat agitation or the signs of agitation, the weight ratio of respectively Dexmedetomidine or a pharmaceutically acceptable salt thereof to the second active agent may generally be in the range from about 1:2 to about 1:2.5; about 1:2.5 to about 1:3; about 1:3 to about 1.3.5 about 1:3.5 to about 1:4; about 1:4 to about 1:4.5, about 1:4.5 to about 1.5; about 1:5 to about 1:10; and about 1:10 to about 1:25. For example, the weight ratio may particularly be between about 1:1 to about 1:5; about 1:5 to about 1:10; about 1:10 to about 1:15; or about 1:15 to about 1:25. Alternatively, the weight ratio of respectively the second active agent to Dexmedetomidine or a pharmaceutically acceptable salt may be in the range of from about 2:1 to about 2.5:1; about 2.5:1 to about 3:1; about 3:1 to about 3.5:1; about 3.5:1 to about 4:1; about 4:1 to about 4.5:1; about 4.5:1 to about 5:1; about 5:1 to about 10:1; and about 10:1 to about 25:1. For example, the weight ratio of respectively the second active agent to Dexmedetomidine or a pharmaceutically acceptable salt thereof may particularly be in the range of from about 1:1 to about 5:1; about 5:1 to about 10.1; about 10:1 to about 15:1; or about 15.1 to about 25:1. It is to be understood that all ranges between the quoted ranges are also covered herein, and constitute further particular aspects of this invention.

### VI. Dosing Regimen

The dosing regimen employed may depend on several factors, such as the type of agitation treated, the severity of the signs, and whether the agitation is due to an underlying medical condition.

Dexmedetomidine or a pharmaceutically acceptable salt thereof may be administered sublingually in any appropriate dose to an animal (e.g.. human). In certain embodiments, the human dose may be from about 3 micrograms to about 100 micrograms (e.g.. about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms (e.g.. about 5, 6, 7, 8, or 9 micrograms), about 10 micrograms, about 12 micrograms, about 14 micrograms, about 15 micrograms, about 16 micrograms, about 18 micrograms, about 20 micrograms, about 30 micrograms, about 50 micrograms). The dose may be administered one or more times a day.

Dexmedetomidine or a pharmaceutically acceptable salt thereof may be administered sublingually in any appropriate dose to a human. In some variations, the human dose may be from about 0.05 micrograms/kg weight of subject to about 1.5 micrograms/kg weight of subject. Examples of suitable dosages include: about 0.1 micrograms/kg to about 1 micrograms/kg, about 0.1 micrograms/kg to about 0.5 micrograms/kg, about 0.1 micrograms/kg to about 0.4 micrograms/kg, about 0.1 micrograms/kg to about 0.3 micrograms/kg, about 0.1 micrograms/kg to about 0.2 micrograms/kg, about 0.07 micrograms/kg, about 0.05 micrograms/kg, about 0.1 micrograms/kg, about 0.2 micrograms/kg, about 0.3 micrograms/kg, about 0.4 micrograms/kg, about 0.5 micrograms/kg, about 0.6 micrograms/kg, about 0.7 micrograms/kg, about 0.8 micrograms/kg, about 0.9 micrograms/kg, about 1.0 micrograms/kg, about 1.1 micrograms/kg, about 1.2 micrograms/kg, about 1.3 micrograms/kg, about 1.4 micrograms/kg, about 1.5 micrograms/kg. The dose may be administered one or more times a day.

### VII. Specific Embodiments of the Invention

Embodiment 1. A method of treating agitation or the signs of agitation in a subject in need thereof comprising administering sublingually to said subject an effective amount of Dexmedetomidine or a pharmaceutically acceptable salt thereof.

Embodiment 2. A method of treating agitation or the signs of agitation in a subject in need thereof comprising administering to said subject an effective amount of Dexmedetomidine or a pharmaceutically acceptable salt thereof, wherein said Dexmedetomidine or a pharmaceutically acceptable salt thereof is sublingually administered at a dosage that treats agitation or the signs of agitation without causing significant sedation.

Embodiment 3. The method according to Embodiment 1 or 2, wherein said dosage of Dexmedetomidine or a pharmaceutically acceptable salt thereof is in range of from about 3micrograms to about 100 micrograms (e.g.. about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms (e.g.. about 5, 6, 7, 8, or 9 micrograms), about 10 micrograms, about 12 micrograms, about 14 micrograms, about 15 micrograms, about 16 micrograms, about 18 micrograms, about 20 micrograms, about 30 micrograms, about 50 micrograms).

Embodiment 4. The method according to Embodiments 1, 2 or 3, wherein said subject is mammal, preferably human.

Embodiment 5 The method according to any one of Embodiments 1 to 4, wherein said agitation is associated with a neurodegenerative disease.

Embodiment 6. The method according to Embodiment 5, wherein said neurodegenerative disease is selected from Alzheimer disease, fronto-temporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, and progressive supranuclear palsy.

Embodiment 7. The method according to any one of Embodiments 1 to 4, wherein the agitation is associated with a neuropsychiatric condition.

Embodiment 8. The method according to Embodiment 7, wherein said neuropsychiatric condition is selected from schizophrenia, bipolar illness (such as mania, disorder), delirium, and depression.

Embodiment 9. The method according to Embodiment 5, wherein the agitation is associated with sundown syndrome in dementia or Alzheimer's disease.

Embodiment 10. A sublingual composition for use in the treatment of agitation or the signs of agitation in a subject in need thereof, wherein said agitation is not perioperative agitation and said sublingual composition comprises an effective amount of Dexmedetomidine or a pharmaceutically acceptable salt thereof and one or more pharmaceutically acceptable carriers/excipients.

Embodiment 11. A sublingual composition for use in the treatment of agitation or the signs of agitation in a subject in need thereof, wherein said agitation is associated with a neurodegenerative disease and said sublingual composition comprises an effective amount of Dexmedetomidine or a pharmaceutically acceptable salt thereof and one or more pharmaceutically acceptable carriers/excipients.

Embodiment 12. A sublingual composition for use in the treatment of agitation or the signs of agitation in a subject in need thereof, wherein said agitation is associated with a neuropsychiatric condition and said sublingual composition comprises an effective amount of Dexmedetomidine or a pharmaceutically acceptable salt thereof and one or more pharmaceutically acceptable carriers/excipients.

Embodiment 13. A sublingual composition for use in the treatment of agitation or the signs of agitation in a subject in need thereof, wherein said agitation is associated with sundown syndrome in dementia or Alzheimer's disease and said sublingual composition comprises an effective amount of Dexmedetomidine or a pharmaceutically acceptable salt thereof and one or more pharmaceutically acceptable carriers/excipients.

Embodiment 14. The sublingual composition according to Embodiment 11, wherein said neurodegenerative disease is selected from the group consisting of Alzheimer disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis Creutzfeldt-Jakob disease, multiple system atrophy, and progressive supranuclear palsy.

Embodiment 15. The sublingual composition according to Embodiment 14, wherein said neurodegenerative disease is selected from dementia, frontotemporal dementia, Alzheimer's disease and Parkinson's disease.

Embodiment 16. The sublingual composition according to Embodiment 12, wherein said neuropsychiatric condition is selected from the group consisting of schizophrenia, bipolar illness (such as mania, disorder), delirium and depression.

Embodiment 17. The sublingual composition according to any one of Embodiments 10 to 16, wherein said composition is selected from a film, wafer, patch, lozenge, gel, spray, tablet, liquid drops or the like.

Embodiment 18. The sublingual composition according to Embodiment 17, wherein the composition is a film.

Embodiment 19. The sublingual composition according to Embodiment 18, wherein the film is mucoadhesive in nature and provides a quick onset of action.

Embodiment 20. The sublingual composition according to any one of Embodiments 10 to 19, wherein Dexmedetomidine or a pharmaceutically acceptable salt thereof is administered at a dosage that treats agitation or the signs of agitation without causing significant sedation.

Embodiment 21. The sublingual composition according to Embodiment 20 wherein the observed level of sedation is not greater than 3 on the Ramsay sedation scale.

Embodiment 22. The sublingual composition according to any one of Embodiments 10 to 21, wherein Dexmedetomidine or a pharmaceutically acceptable salt thereof is administered to said subject (e g. human) at a dosage in the range of from about 3 micrograms to about 100 micrograms (e.g.. about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms (e.g.. about 5, 6, 7, 8, or 9 micrograms), about 10 micrograms, about 12 micrograms, about 14 micrograms, about 15 micrograms, about 16 micrograms, about 18 micrograms, about 20 micrograms, about 30 micrograms, about 50 micrograms).

Embodiment 23. A method of treating agitation or the signs of agitation in a subject in need thereof, the method comprises sublingually administering to said subject an effective amount of Dexmedetomidine or a pharmaceutically acceptable salt thereof, wherein the agitation is not perioperative agitation.

Embodiment 24. The method according to Embodiment 23, wherein the agitation is associated with a neurodegenerative disease and/or neuropsychiatric condition.

Embodiment 25. The method according to Embodiment 24, wherein the neurodegenerative disease is selected from the group consisting of Alzheimer disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, progressive supranuclear palsy or other related neurodegenerative disorder.

Embodiment 26. The method according to Embodiment 24, wherein the neuropsychiatric condition is selected from the group consisting of schizophrenia, bipolar illness (e.g.. bipolar disorder or bipolar mania), delirium and depression.

Embodiment 27. The method according to any one of Embodiments 23 to 26, wherein agitation or the signs of agitation are treated effectively without causing significant sedation.

Embodiment 28. The method according to any one of Embodiments 23 to 27, wherein Dexmedetomidine or a pharmaceutically acceptable salt thereof is administered in form of a film, wafer, patch, lozenge, gel, spray, tablet, liquid drops or the like.

Embodiment 29. A method of treating of agitation or the signs of agitation in a subject in need thereof, wherein said agitation is associated with an OPD/IPD procedure (e.g.. MRI, CT or CAT scan, lumbar puncture, bone marrow aspiration/biopsy, tooth extraction or other dental procedures), and said method comprises administering to said subject sublingually an effective amount of Dexmedetomidine or a pharmaceutically acceptable salt thereof.

Embodiment 30. A method of treating of agitation or the signs of agitation in a subject

in need thereof, wherein said agitation is associated with an alcohol and substance abuse withdrawal and said method comprises administering to said subject sublingually an effective amount of Dexmedetomidine or a pharmaceutically acceptable salt thereof.

Embodiment 31. The method according to Embodiment 29 or 30, wherein said Dexmedetomidine or a pharmaceutically acceptable salt thereof is sublingually administered at a dosage that treats said agitation or the signs of agitation without causing significant sedation.

Embodiment 32. The method according to Embodiment 31, wherein said dosage of Dexmedetomidine or a pharmaceutically acceptable salt thereof is in range of from about 3 micrograms to about 100 micrograms (e.g.. about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms (e.g.. about 5, 6, 7, 8, or 9 micrograms), about 10 micrograms, about 12 micrograms, about 14 micrograms, about 15 micrograms, about 16 micrograms, about 18 micrograms, about 20 micrograms, about 30 micrograms, about 50 micrograms).

Embodiment 33. The composition or method according to any preceding Embodiment, wherein Dexmedetomidine or a pharmaceutically acceptable salt thereof is administered once, twice or thrice daily or on an “as needed” basis.

Embodiment 34. The composition or method according to any preceding Embodiment, wherein Dexmedetomidine or a pharmaceutically acceptable salt thereof is administered in a manner that produces a therapeutic effect in less than about 60 minutes, particularly within about 30 seconds to about 30 minutes.

Embodiment 35. A method of treating agitation or the signs of agitation in a subject in need thereof comprising administering sublingually to said subject an effective amount of an alpha-2 adrenergic agonist or a pharmaceutically acceptable salt thereof.

Embodiment 36. A method of treating agitation or the signs of agitation in a subject in need thereof comprising administering to said subject an effective amount of an alpha-2 adrenergic agonist or a pharmaceutically acceptable salt thereof, wherein said alpha-2 adrenergic agonist is sublingually administered at a dosage that treats agitation or the signs of agitation without causing significant sedation.

Embodiment 37. The method according to Embodiment 35 or 36, wherein said dosage of alpha-2 adrenergic agonist is in range of from about 3 micrograms to about 100 micrograms (e.g.. about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms (e.g.. about 5, 6, 7, 8, or 9 micrograms), about 10 micrograms, about 12 micrograms, about 14 micrograms, about 15 micrograms, about 16 micrograms, about 18 micrograms, about 20 micrograms, about 30 micrograms, about 50 micrograms).

Embodiment 38. The method according to Embodiments 35, 36 or 37, wherein said subject is mammal, preferably human.

Embodiment 39. The method according to any one of Embodiments 35 to 38, wherein said agitation is associated with a neurodegenerative disease.

Embodiment 40. The method according to Embodiment 39, wherein said neurodegenerative disease is selected from Alzheimer disease, fronto-temporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, and progressive supranuclear palsy.

Embodiment 41. The method according to any one of Embodiments 35 to 38, wherein the agitation is associated with a neuropsychiatric condition.

Embodiment 42. The method according to Embodiment 41, wherein said neuropsychiatric condition is selected from schizophrenia, bipolar illness (such as mania, disorder), delirium, and depression.

Embodiment 43. The method according to Embodiment 39, wherein the agitation is associated with sundown syndrome in dementia or Alzheimer's disease.

Embodiment 44. The method according to any one of Embodiments 35 to 38, wherein said agitation is associated with an OPD/IPD procedure (e.g.. MRI, CT or CAT scan, lumbar puncture, bone marrow aspiration/biopsy, tooth extraction or other dental procedures).

Embodiment 45. The method according to any one of Embodiments 35 to 38, wherein said agitation is associated with an alcohol and substance abuse withdrawal

Embodiment 46. The method according to any one of Embodiments 35 to 38, wherein said alpha-2 adrenergic agonist include, but is not limited to, Clonidine, Guanfacine, Guanabenz, Guanoxabenz, Guanethidine, Xylazine, Tizanidine, Medetomidine, Dexmedetomidine, Methyldopa, Methylnorepinephrine, Fadolmidine Iodoclonidine, Apraclonidine, Detomidine, Lofexidine, Amitraz, Mivazerol, Azepexol, Talipexol, Rilmenidine, Naphazoline, Oxymetazoline, Xylometazoline, Tetrahydrozoline, Tramazoline, Talipexole, Romifidine, propylhexedrine, Norfenefrine, Octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof.

Embodiment 47. A method of treating, preventing or reducing the symptoms of agitation in a subject in need thereof, wherein said agitation is associated with neurodegenerative diseases and said method comprises administering to said subject a therapeutically effective amount of Dexmedetomidine, or a pharmaceutically acceptable salt thereof.

Embodiment 48. A method of treating, preventing or reducing the symptoms of agitation in a subject in need thereof, wherein said agitation is associated with sundowning syndrome in dementia or Alzheimer's disease and said method comprises: administering to said subject a therapeutically effective amount of Dexmedetomidine, or a pharmaceutically acceptable salt thereof.

Embodiment 49. A method of treating, preventing or reducing the symptoms of agitation in a subject in need thereof, wherein said agitation is associated with schizophrenia, bipolar disorder, bipolar mania, delirium, intoxication and other neuropsychiatric conditions and said method comprises: administering to said subject a therapeutically effective amount of Dexmedetomidine, or a pharmaceutically acceptable salt thereof.

Embodiment 50. The method as embodied in embodiment 47, wherein said neurodegenerative disease is Dementia, Frontotemporal dementia, Alzheimer's disease, Parkinson's disease, Multiple system atrophy, Creutzfeldt-jakob disease, Corticobasal degeneration, Gerstmannstraussler-scheinker syndrome, Huntington disease, Fatal familial insomnia, Cushing's syndrome, Hypercortisolism, Neurofibromatosis type 1, Norrie disease, Progressive supranuclear palsy, Hereditary spastic paraplegia, Alpers syndrome, Smith-Lemli Opitz syndrome, Fragile x syndrome, Mulvihill-smith syndrome, Transmissible spongiform encephalopathy, Morvan syndrome, Morvan's fibrillary chorea, Peripheral nerve hyperexcitability and Agrypnia Excitata.

Embodiment 51. The method as embodied in embodiment 50, wherein said neurodegenerative disease is preferably Dementia, Frontotemporal dementia, Alzheimer's disease, Parkinson's disease.

Embodiment 52. The method as embodied in embodiments 47 to 49, wherein dexmedetomidine, or a pharmaceutically acceptable salt thereof is administered to said subject by oromucosal route including sublingual or buccal route in the form of pharmaceutical composition.

Embodiment 53. The method as embodied in embodiment 52, wherein dexmedetomidine, or a pharmaceutically acceptable salt thereof is administered to said subject by sublingual route.

Embodiment 54. The method as embodied in embodiments 47 to 49, wherein dexmedetomidine, or a pharmaceutically acceptable salt thereofis administered to said subject (i.e. Human) is in a range of about 1 μg to about 105 μg, preferably 3 μg to 100 μg, more preferably 5 μg to 50 μg.

Embodiment 55. A method of treating, preventing or reducing the symptoms of agitation in a subject in need thereof wherein said agitation is associated with neurodegenerative diseases and said method comprises administering to said subject a pharmaceutical composition comprising a therapeutically effective amount of dexmedetomidine, or a pharmaceutically acceptable salt thereof and pharmaceutical acceptable excipient(s).

Embodiment 56. A method of treating, preventing or reducing the symptoms of agitation in a subject in need thereof wherein said agitation is associated with schizophrenia, bipolar disorder, bipolar mania, delirium, post-traumatic stress disorder, alcohol and substance abuse withdrawal, intoxication and other neuropsychiatric conditions and said method comprises administering to said subject a pharmaceutical composition comprising a therapeutically effective amount of dexmedetomidine, or a pharmaceutically acceptable salt thereof and pharmaceutical acceptable excipient(s).

Embodiment 57. A method of treating, preventing or reducing the symptoms of agitation in a subject in need thereof wherein said agitation is associated with sundowning syndrome in dementia or Alzheimer's disease and said method comprises administering to said subject a pharmaceutical composition comprising a therapeutically effective amount of dexmedetomidine, or a pharmaceutically acceptable salt thereof and pharmaceutical acceptable excipient(s).

Embodiment 58. A pharmaceutical composition for use in the treatment, prevention or reduction in the symptoms of agitation in a subject in need thereof wherein said chronic agitation is associated with neurodegenerative diseases and said pharmaceutical composition comprises dexmedetomidine, or a pharmaceutically acceptable salt thereof and pharmaceutical acceptable excipient(s).

Embodiment 59. A pharmaceutical composition for use in the treatment, prevention or reduction in the symptoms of agitation in a subject in need thereof wherein said agitation is associated with schizophrenia, bipolar disorder, bipolar mania, delirium, intoxication and other neuropsychiatric conditions and said pharmaceutical composition comprises dexmedetomidine, or a pharmaceutically acceptable salt thereof and pharmaceutical acceptable excipient(s).

Embodiment 60. A pharmaceutical composition for use in the treatment, prevention or reduction in the symptoms of agitation in a subject in need thereof wherein said agitation is associated with sundowning syndrome in dementia or Alzheimer's disease and said pharmaceutical composition comprises dexmedetomidine, or a pharmaceutically acceptable salt thereof and pharmaceutical acceptable excipient(s)

Embodiment 61. The method/composition as embodied in embodiments 55 and 58, wherein said neurodegenerative disease is selected from Frontotemporal dementia, Dementia, Alzheimer's disease, Parkinson's disease Multiple system atrophy, Creutzfeldt-jakob disease, Corticobasal degeneration, Gerstmannstraussler-scheinker syndrome, Huntington disease, Fatal familial insomnia, Cushing's syndrome, Hypercortisolism, Neurofibromatosis type 1, Norrie disease, Progressive supranuclear palsy, Hereditary spastic paraplegia, Alpers syndrome, Smith-Lemli Opitz syndrome, Fragile x syndrome, Mulvihill-smith syndrome, Transmissible spongiform encephalopathy, Morvan syndrome, Morpervan's fibrillary chorea, Peripheral nerve hyperexcitability and Agryphia Excitata.

Embodiment 62. The method/composition as embodied in embodiment 61, wherein said neurodegenerative disease is preferably Dementia, Frontotemporal dementia, Alzheimer's disease, Parkinson's disease

Embodiment 63. The method/composition as embodied in embodiments 55 to 60, wherein said pharmaceutical composition is administered to said subject by an oromucosal route including sublingual or buccal route in the form of pharmaceutical composition.

Embodiment 64. The method/composition as embodied in embodiment 63, wherein said pharmaceutical composition is a tablet, film, gel etc.

Embodiment 65. The method/composition as embodied in embodiments 55 to 60, wherein Dexmedetomidine, or a pharmaceutically acceptable salt thereof is administered to said subject (i.e. Human) is in a range of about 1 μg to about 105 μg, preferably about 3 μg to 100 μg, more preferably about 5 μg to 50 μg and most preferably less than 10 μg.

Embodiment 66. Use of dexmedetomidine, or a pharmaceutically acceptable salt thereof composition for treating a subject suffering from agitation associated with neurodegenerative disease.

Embodiment 67. Use of dexmedetomidine, or a pharmaceutically acceptable salt thereof composition for treating a subject suffering from agitation associated with neuropsychiatric conditions including schizophrenia, bipolar disorder, bipolar mania, delirium, intoxication and other neuropsychiatric conditions

Embodiment 68. Use of dexmedetomidine, or a pharmaceutically acceptable salt thereof composition for treating a subject suffering from agitation associated with sundowning syndrome in dementia or Alzheimer's disease.

Embodiment 69. The use as embodied in embodiment 66, wherein neurodegenerative disease is selected from Frontotemporal dementia, dementia, Alzheimer's disease, Parkinson's disease, Multiple system atrophy, Creutzfeldt-jakob disease, Corticobasal degeneration, Gerstmannstraussler-scheinker syndrome, Huntington disease, Fatal familial insomnia, Cushing's syndrome, Hypercortisolism, Neurofibromatosis type 1, Norrie disease, Progressive supranuclear palsy, Hereditary spastic paraplegia, Alpers syndrome, Smith-Lemli Opitz syndrome, Fragile x syndrome, Mulvihill-smith syndrome, Transmissible spongiform encephalopathy, Morvan syndrome, Morvan's fibrillary chorea, Peripheral nerve hyperexcitability and Agryphia Excitata.

Embodiment 70. The use as embodied in embodiment 69, wherein said neurodegenerative disease is preferably Dementia, Frontotemporal dementia, Alzheimer's disease, Parkinson's disease

Embodiment 71. A sublingual film composition of dexmedetomidine, or a pharmaceutically acceptable salt thereof for use in the treatment of agitation associated with neurodegenerative disease.

Embodiment 72. A sublingual film composition of dexmedetomidine, or a pharmaceutically acceptable salt thereof for use in the treatment of agitation associated with neuropsychiatric conditions such as bipolar disorder, schizophrenia. bipolar mania. Delirium, intoxication and other neuropsychiatric conditions.

Embodiment 73. A sublingual film composition of dexmedetomidine, or a pharmaceutically acceptable salt thereof for use in the treatment of agitation associated with sundowning syndrome in dementia or Alzheimer's disease.

Embodiment 74. A method of treating, preventing or reducing the symptoms of aggression in a subject in need thereof and said method comprises administering to said subject a pharmaceutical composition comprising a therapeutically effective amount of dexmedetomidine, or a pharmaceutically acceptable salt thereof and pharmaceutical acceptable excipient(s).

Embodiment 75. A method of treating, preventing or reducing the symptoms of agitation in a subject in need thereof, the method comprises administering to said subject dexmedetomidine, or a pharmaceutically acceptable salt thereof through sublingual film in a dose range of 1 μg to 105 μg, preferably about 3 μg to 100 μg and more preferably about 5 μg to 50 μg, more preferably about 5 μg to 20 μg, most preferably less than 10 μg.

Embodiment 76. The method as embodied in embodiment 75, wherein the agitation is acute agitation.

Embodiment 77. The method as embodied in embodiment 75, wherein the agitation is associated with an illness including neurodegenerative disease and/or neuropsychiatric conditions.

Embodiment 78. The method as embodied in embodiment 77, wherein the neurodegenerative disease is selected from the group consisting of neurodegenerative disease Dementia, Frontotemporal dementia, Alzheimer's disease, Parkinson's disease, Multiple system atrophy, Creutzfeldt-jakob disease, Corticobasal degeneration, Gerstmannstraussler-scheinker syndrome, Huntington disease, Fatal familial insomnia, Cushing's syndrome, Hypercortisolism, Neurofibromatosis type 1, Norrie disease, Progressive supranuclear palsy, Hereditary spastic paraplegia, Alpers syndrome, Smith-Lemli Opitz syndrome, Fragile X syndrome, Mulvihill-smith syndrome, Transmissible spongiform encephalopathy, Morvan syndrome, Morvan's fibrillary chorea, Peripheral nerve hyperexcitability and Agrypnia Excitata, sundowning syndrome in dementia or sundowning syndrome in Alzheimer's disease, preferably Dementia, Frontotemporal dementia, Alzheimer's disease or Parkinson's disease.

Embodiment 79. The method as embodied in embodiment 77, wherein the neuropsychiatric conditions is selected from the group consisting of schizophrenia, bipolar disorder, bipolar mania, delirium post-traumatic stress disorder, alcohol and substance abuse withdrawal or intoxication.

Embodiment 80. The method embodied in embodiment 75, wherein dexmedetomidine, or a pharmaceutically acceptable salt thereof is administered to the said subject in a dose ranging from about 1 μg to about 105 μg; preferably from about 3 μg to about 100 μg, more preferably about 5 μg to about 50 μg, most preferably about 5 μg to about 20 μg.

Embodiment 81. A sublingual film comprising about 5 μg to about 100 μg of dexmedetomidine, or a pharmaceutically acceptable salt thereof for use in the treatment of agitation in a subject.

Embodiment 82. The sublingual film as embodied in embodiment 81, wherein the agitation is acute agitation.

Embodiment 83. The sublingual film as embodied in embodiment 82, wherein the agitation is associated with an illness including neurodegenerative disease and/or neuropsychiatric conditions.

Embodiment 84. The sublingual film as embodied in embodiment 83, wherein the neurodegenerative disease is selected from the group consisting of neurodegenerative disease Dementia, Frontotemporal dementia, Alzheimer's disease, Parkinson's disease, Multiple system atrophy, Creutzfeldt-jakob disease, Corticobasal degeneration, Gerstmannstraussler-scheinker syndrome, Huntington disease, Fatal familial insomnia, Cushing's syndrome, Hypercortisolism, Neurofibromatosis type 1, Norrie disease, Progressive supranuclear palsy, Hereditary spastic paraplegia, Alpers syndrome, Smith-LemliOpitz syndrome, Fragile X syndrome, Mulvihill-smith syndrome, Transmissible spongiform encephalopathy, Morvan syndrome, Morvan's fibrillary chorea, Peripheral nerve hyperexcitability, Agrypnia Excitata, sundowning syndrome in dementia or sundowning syndrome in Alzheimer's disease, preferably Dementia, Frontotemporal dementia, Alzheimer's disease or Parkinson's disease.

Embodiment 85. The sublingual film as embodied in embodiment 83, wherein the neuropsychiatric conditions is selected from the group consisting of schizophrenia, bipolar disorder, bipolar mania, delirium post-traumatic stress disorder, alcohol and substance abuse withdrawal or intoxication.

Embodiment 86. The sublingual film as embodied in embodiment 81, wherein the film comprises dexmedetomidine, or a pharmaceutically acceptable salt thereof ranging from about 1 μg to about 105 μg; preferably from about 3 μg to about 100 μg, more preferably about 5 μg to about 50 μg and most preferably about 5 μg to about 20 μg and less than 10 μg.

Embodiment 87. Use of dexmedetomidine, or a pharmaceutically acceptable salt thereof for treating, preventing or reducing the symptoms of agitation in a subject in need thereof, wherein the agitation or aggression is not perioperative agitation.

Embodiment 88. The use as embodied in embodiment 87, wherein the agitation is associated with an illness including neurodegenerative disease and/or neuropsychiatric conditions.

Embodiment 89. The use as embodied in embodiment 88, wherein the neurodegenerative disease is selected from the group consisting of neurodegenerative disease Dementia, Frontotemporal dementia, Alzheimer's disease, Parkinson's disease, Multiple system atrophy, Creutzfeldt-jakob disease, Corticobasal degeneration, Gerstmannstraussler-scheinker syndrome, Huntington disease, Fatal familial insomnia, Cushing's syndrome, Hypercortisolism, Neurofibromatosis type 1, Norrie disease, Progressive supranuclear palsy, Hereditary spastic paraplegia, Alpers syndrome, Smith-LemliOpitz syndrome, Fragile X syndrome, Mulvihill-smith syndrome, Transmissible spongiform encephalopathy, Morvan syndrome, Morvan's fibrillary chorea, Peripheral nerve hyperexcitability, Agrypnia Excitata, sundowning syndrome in dementia or sundowning syndrome in Alzheimer's disease, preferably Dementia, Frontotemporal dementia, Alzheimer's disease and Parkinson's disease.

Embodiment 90. The use as embodied in embodiment 88, wherein the neuropsychiatric conditions are selected from the group consisting of schizophrenia, bipolar disorder, bipolar mania, delirium post-traumatic stress disorder, alcohol and substance abuse withdrawal and intoxication.

Embodiment 91. The use as embodied in embodiment 87, wherein the agitation is acute agitation.

Embodiment 92. The use as embodied in embodiment 87, wherein Dexmedetomidine, or a pharmaceutically acceptable salt thereof is administered to said subject by oromucosal route preferably by sublingual or buccal route

Embodiment 93. The use as embodied in embodiment 92, wherein Dexmedetomidine, or a pharmaceutically acceptable salt thereof is administered in form of pharmaceutical composition selected from the group comprising of tablet, film and gel.

Embodiment 94. The use as embodied in embodiment 87, wherein Dexmedetomidine, or a pharmaceutically acceptable salt thereof is administered to the said subject in a dose ranging from about 1 μg to about 105 μg: preferably from about 3 μg to about 100 μg, more preferably about 5 μg to about 50 μg, most preferably about 5 μg to about 20 μg and most preferably less than 10 μg.

Embodiment 95. A method of treating, preventing or reducing the symptoms of agitation in a subject in need thereof, the method comprises administering to said subject a therapeutically effective amount of dexmedetomidine, or a pharmaceutically acceptable salt thereof by sublingual route, wherein the agitation is not perioperative agitation.

Embodiment 96. A sublingual pharmaceutical composition for treating, preventing or reducing the symptoms of agitation in a subject in need thereof, the composition comprises a therapeutically effective amount of dexmedetomidine, or a pharmaceutically acceptable salt thereof and one or more pharmaceutical excipient, wherein the agitation is not perioperative agitation.

Embodiment 97. A sublingual use of dexmedetomidine, or a pharmaceutically acceptable salt thereof for treating, preventing or reducing the symptoms of agitation in a subject in need thereof, wherein the agitation is not perioperative agitation.

Embodiment 98. A sublingual composition of dexmedetomidine, or a pharmaceutically acceptable salt thereof for use in the treatment of agitation associated with neurodegenerative disease.

Embodiment 99. A sublingual composition of dexmedetomidine, or a pharmaceutically acceptable salt thereof for use in the treatment of agitation associated with bipolar disorder, schizophrenia. bipolar mania. Delirium, intoxication and other neuropsychiatric conditions.

Embodiment 100. A sublingual composition of dexmedetomidine, or a pharmaceutically acceptable salt thereof for use in the treatment of agitation associated with sundowning syndrome in dementia or Alzheimer's disease.

Embodiment 101. A method of treating, preventing or reducing the symptoms of agitation which is other than perioperative agitation in a subject in need thereof, the method comprises administering to said subject a therapeutically effective amount of dexmedetomidine, or a pharmaceutically acceptable salt thereof by sublingual route.

Embodiment 102. A sublingual film for treating, preventing or reducing the symptoms of agitation which is other than perioperative agitation in a subject in need thereof, the method comprises administering to said subject a therapeutically effective amount of dexmedetomidine, or a pharmaceutically acceptable salt thereof.

Embodiment 103. A method of treating, preventing or reducing the symptoms of aggression in a subject in need thereof and said method comprises administering to said subject a pharmaceutical composition comprising a therapeutically effective amount of dexmedetomidine, or a pharmaceutically acceptable salt thereof and pharmaceutical acceptable excipient(s).

Embodiment 104. The method as embodied in embodiment 96, wherein the agitation is associated with an illness including neurodegenerative disease and/or neuropsychiatric conditions.

Embodiment 105. The method as embodied in embodiment 102, wherein the neurodegenerative disease is selected from the group consisting of neurodegenerative disease Dementia, Frontotemporal dementia, Alzheimer's disease, Parkinson's disease, Multiple system atrophy, Creutzfeldt-jakob disease, Corticobasal degeneration, Gerstmannstraussler-scheinker syndrome, Huntington disease, Fatal familial insomnia, Cushing's syndrome, Hypercortisolism, Neurofibromatosis type 1, Norrie disease, Progressive supranuclear palsy, Hereditary spastic paraplegia, Alpers syndrome, Smith-LemliOpitz syndrome, Fragile X syndrome, Mulvihill-smith syndrome, Transmissible spongiform encephalopathy, Morvan syndrome, Morvan's fibrillary chorea, Peripheral nerve hyperexcitability, Agryphia Excitata, sundowning syndrome in dementia or sundowning syndrome in Alzheimer's disease, preferably Dementia, Frontotemporal dementia, Alzheimer's disease or Parkinson's disease.

Embodiment 106. The method as embodied in embodiment 102, wherein the neuropsychiatric conditions is selected from the group consisting of schizophrenia, bipolar disorder, bipolar mania, delirium post-traumatic stress disorder, alcohol and substance abuse withdrawal or intoxication.

Embodiment 107. The method as embodied in embodiment 96, wherein dexmedetomidine, or a pharmaceutically acceptable salt thereof is administered to said subject by oromucosal route preferably by sublingual or buccal route.

Embodiment 108. The method as embodied in embodiment 105, wherein dexmedetomidine, or a pharmaceutically acceptable salt thereof is administered in form of pharmaceutical composition selected from the group comprising of tablet, film or gel.

Embodiment 109. A method of treating, preventing or reducing the symptoms of agitation in a subject in need thereof wherein said agitation is associated with other conditions such as OPD/IPD Procedures like MRI, CT or CAT Scan, Lumbar Puncture, Bone marrow aspiration/biopsy, Tooth extraction or other dental procedures and said method comprises administering to said subject a pharmaceutical composition comprising a therapeutically effective amount of dexmedetomidine, or a pharmaceutically acceptable salt thereof and pharmaceutical acceptable excipient(s).

Embodiment 110. The methods/composition as embodied in the preceding embodiments, wherein the pharmaceutical composition includes intranasal formulations, parenteral formulations, oral formulations, transdermal formulations, liposomal formulations and the like.

The Embodiments hereinabove are not intended to be limiting, and, in practicing the present invention, alternative or additional Embodiments may be provided.

### VIII. Examples

The following Examples are intended to be illustrative and not limiting:

### Example 1

### Formulation 1: Sub-Lingual Tablet

TABLE 1
Composition for a typical Sub-lingual tablet formulation
used for sublingual delivery

| Ingredients | Quantity | Ranges |
|---|---|---|
| Dexmedetomidine HCl | 50 | |
| (equivalent to base) | micrograms | |
| Povidone | 5.0 mg | 1.0-10.0% |
| Croscarmellose Sodium | 7.0 mg | 5-15% |
| Sucralose | 1.0 mg | 0.05-3.0% |
| Magnesium Stearate | 0.75 mg | 0.1-2.0% |
| Talc | 0.75 mg | 0.1-2.0% |
| Mannitol | q.s 75.0 mg | q.s. 100% |
| Water | q.s | |

### Manufacturing Process

Dexmedetomidine hydrochloride and excipients such as binder and sweetener are dissolved/dispersed into a pharmaceutically acceptable solvent (preferably water) and this solution is used to granulate the sifted blend of all other ingredients except lubricant and glidant in suitable mixer/granulator. The granules are then dried in a fluid-bed drier or other suitable one such as tray drier. The dried granules are then sized appropriately in quadro-co-mill or multi-mill. The sized granules are then loaded into a suitable blender such as V-blender and lubricated with Magnesium stearate and Talc and then the final lubricated blend is then used for compressing into tablets of specific dimensions using appropriate tooling.

### Formulation 2: Sub-Lingual Film

TABLE 2
Composition for a typical Sub-lingual film formulation
used for sublingual delivery

| Ingredients | Quantity | Ranges |
|---|---|---|
| Dexmedetomidine HCl | 50 | |
| (equivalent to base) | micrograms | |
| Polyethylene oxide | 5.0 mg | 3-25% |
| Polyethylene Glycol | 5.0 mg | 3-25% |
| Sucralose | 0.2 mg | 0.05-3.0% |
| Flavoring agent | q.s. | 0.01-1.0% |
| Coloring agent | q.s. | 0.01-1.0% |
| Povidone | q.s. 50 mg | q.s. 100% |

### Manufacturing Process

Dexmedetomidine hydrochloride along with film forming polymers and other excipients are dissolved/dispersed into a pharmaceutically acceptable solvent (preferably water) and the resulting solution is then coated (spread/cast) on an inert backing layer. Dexmedetomidine hydrochloride containing polymeric layer is further dried, separated and cut into suitable sizes using appropriate die/tools and then packed as per the requirement.

### Formulation 3: Sub-Lingual Spray

TABLE 3
Composition for a typical Sub-lingual spray formulation
used for sublingual delivery

| Ingredients | Quantity | Ranges |
|---|---|---|
| Dexmedetomidine HCl | 50 | |
| (equivalent to base) | micrograms | |
| Propylene Glycol | 10 μL | 1.0-40.0% |
| Alcohol | 5 μL | 1.0-40.0% |
| Citric acid | 0.2 mg | 0.1-10% |
| Peppermint Oil | 1 μL | 0.05-3.0% |
| Purified water | q.s. 100 μL | q.s. 100% |

### Manufacturing Process

Dexmedetomidine hydrochloride along with all other excipients are mixed in a suitable order. The resulting solution/dispersion is then filled into spray canisters using appropriate tooling. They are further processed with Metered nozzles so that a specified amount of Dexmedetomidine is delivered after actuation each time.

### Formulation 4: Sub-Lingual Liquid Drops

TABLE 4
Composition for typical Sub-lingual liquid
drops used for sublingual delivery

| Ingredients | Quantity |
|---|---|
| Dexmedetomidine HCl | 10 mg |
| (equivalent to base) | |
| Normal saline | q.s |
| (0.9% Sodium Chloride) | |

### Manufacturing Process

Dexmedetomidine hydrochloride ((Catalogue No. SML0956) was dissolved in Normal saline in order to yield the concentration of 1 mg/ml of the sublingual drops.

### Example 2

Evaluate the effect of sublingual and intravenous administration of Dexmedetomidine hydrochloride in rat ‘resident-intruder’ model of agitation or aggression at varying dosages.

The resident-intruder model is an established preclinical model of aggression and agitation, and allows spontaneous and natural expression of both offensive aggression/agitation and defensive behavior in laboratory rodents in a semi natural laboratory setting. When rodents are exposed to a novel male in their home cage environment, they perceive the novel male animal as an “intruder” and demonstrate a repertoire of defensive behaviors such as ano-genital sniffing, chasing, biting and attacking (Nelson et al., ILAR Journal (2000) 41(3):153-162).

### Materials and Methods

Animals: 12-13 week old male Wistar rats weighing 380-400 g were used as resident males. 7-8 weeks old male rats weighing 280-300 g were used as the “intruder”. Resident rats were housed with female rats for 8 days to establish territoriality. The intruder rats were boused in groups of 3 with other male rats of similar age/body weight. All animals were maintained in a controlled environment with 22±3° C. temperature, 50±20% humidity, a light/dark cycle of 12hours each and 15-20 fresh air changes per hour and had access to food and water ad-libitum. All animal experiments were conducted in accordance with the guidelines of the Committee for the Purpose of Control and Supervision of Experiments on Animals (CPCSEA), Government of India the Association for Assessment and Accreditation of Laboratory Animal Care international (AAALAC)

Formulation tested: The required quantity of Formulation 4 of dexmedetomidine hydrochloride was weighed and serial dilutions were made to obtain respective doses as per the Table 5. Dilutions were prepared fresh every day prior to dosing using 0.9% normal saline from the formulation 4 for the entire study.

Experimental Procedure: Following acclimatization for a period of 3-5 days, each resident male rat was housed with a female rat for 8 days. On day 8, basal aggression in the resident males was tested by exposing them to an “intruder rat” for 10 minutes. Only animals that demonstrated aggression in this basal aggression test were used for the study. These animals were then randomized using body weight stratification method. The weight variation of the animals did not exceed 20% of the mean body weight in a group at the time of randomization. Animals were housed with the female rat for an additional day. On day 9, the resident animal was paired with intruder animal of an appropriate bodyweight such that the body weight of the resident was always higher than the intruder. This was to facilitate dominant, aggressive behavior in the resident animals. After randomization, animals were assigned a permanent number. Cages were identified by cage cards indicating the study number, study code, group number, sex, dose, cage number and animal number details.

Resident male rats were dosed with different doses of Dexmedetomidine hydrochloride (Dex) 15 minutes prior to the behavioral testing either sublingually or intravenously (Table 5). For sublingual dosing, the rats were held in one hand and using a blunt spatula the tongue was moved to one side of the mouth. Dexmedetomidine hydrochloride was then administered sublingually as liquid drops at specific concentration using a micropipette and allowed to be absorbed for a duration of 50-60 seconds. Diazepam was used as a reference compound and was dosed intraperitoneally. Vehicle controls were treated with 0.9% saline administered sublingually or intravenously. Normal controls (NC) did not received any treatment.

The behavior of the resident rat was recorded using an overhead video camera for 15 minutes and offline behavioral analysis was done using the Noldus Ethovision XT software. To distinguish the resident rat from the intruder rat in the video recording, the intruder rat was marked with non-toxic paint. For analysing the potential effects of Dexmedetomidine hydrochloride on agitation, we quantified various behavioral parameters such as anogenital sniffing, chasing, biting, attacking and latency to attack as well as neutral behavioral parameters such as exploration grooming, and immobile quiet time.

TABLE 5
Efficacy Study: Drug treatment groups

| | | | Cohort 2 |
|---|---|---|---|
| | | Cohort 1 | (Intravenous dosing- |
| | | (Sublingual dosing- | Dexmedetomidine |
| Group | No. of | Formulation 4 adjusted to | hydrochloride in |
| No. | animals | following doses) | water or Normal saline) |
| 1 | 8 | Normal Control | |
| 2 | 8 | Vehicle control | Vehicle control |
| 3 | 8 | Dexmedetomidine hydrochloride | Dexmedetomidine |
| | | (0.5 μg/kg) | hydrochloride (0.5 μg/kg) |
| 4 | 8 | Dexmedetomidine hydrochloride | Dexmedetomidine |
| | | (1.0 μg/kg) | hydrochloride (1.0 μg/kg) |
| 5 | 8 | Dexmedetomidine hydrochloride | Dexmedetomidine |
| | | (1.5 μg/kg) | hydrochloride (1.5 μg/kg) |
| 6 | 8 | Dexmedetomidine hydrochloride | Dexmedetomidine |
| | | (3.0 μg/kg) | hydrochloride (3.0 μg/kg) |

| | | |
|---|---|---|
| 7 | 8 | Diazepam (3 mg/kg, i.p.) |

Statistical Analysis: Statistical analysis was performed using validated statistical software (GraphPad Prism 6). Data is represented as Mean±SEM. One-way ANOVA (analysis of variance) followed by “Dunnett's Multiple Comparison Test” at 95% confidence interval was applied for comparison of the relevant groups. p<0.05 was considered significant.

Results: The present study was performed to evaluate the effect of sublingually/intravenously administered different doses of Dexmedetomidine hydrochloride on agitated behavior in a rat resident-intruder model of aggression and agitation behavior.

### Effect of Sublingually/Intravenously Administered Dexmedetomidine Hydrochloride on Aggressive/Agitative Behavior in the Rat Resident Intruder Model

The rats demonstrate a variety of defensive agitated behaviors such as anogenital sniffing, chasing, biting and attacking (indices of agitative and aggressive behavior) when exposed to a novel male in their home cage environment. The non-resident male is perceived as intruder and the resident male gets agitated and attacks the intruder male to protect their home territory. In the present experiments, vehicle treated rats demonstrated a wide repertoire of aggressive behaviors and the intruder rat was subjected to anogenital sniffing, attack, chasing and biting by the resident or dominant rat.

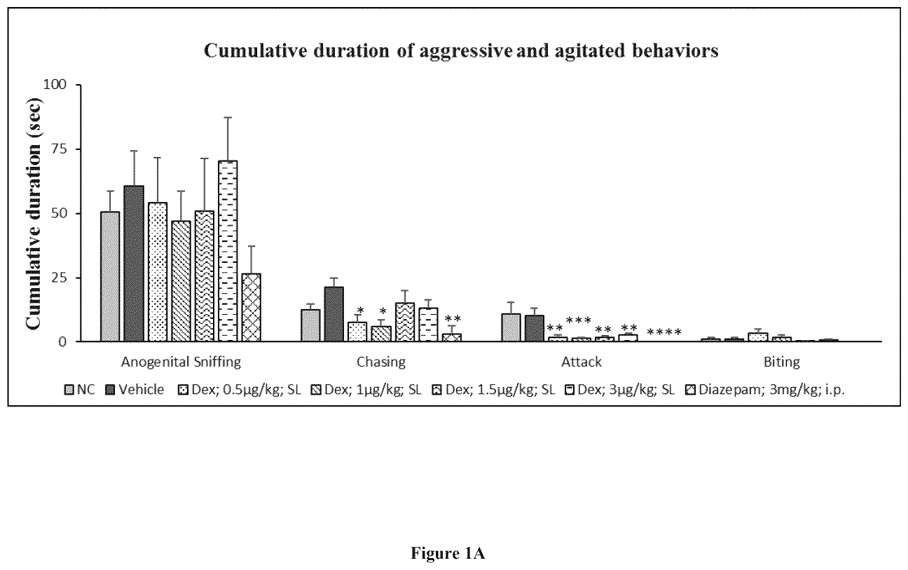

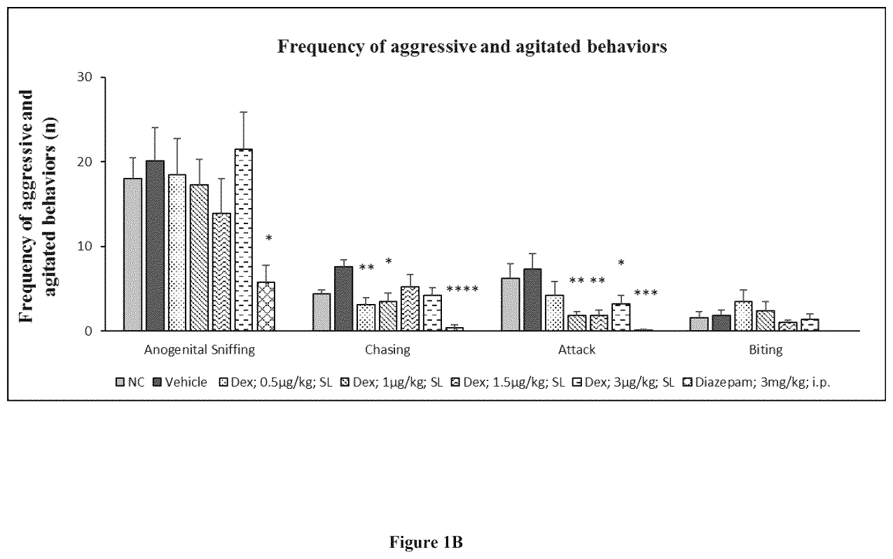

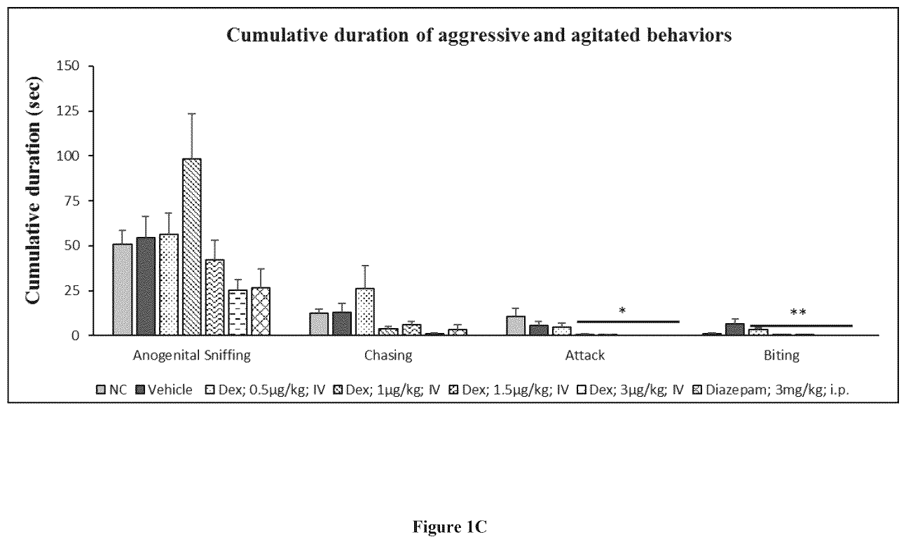

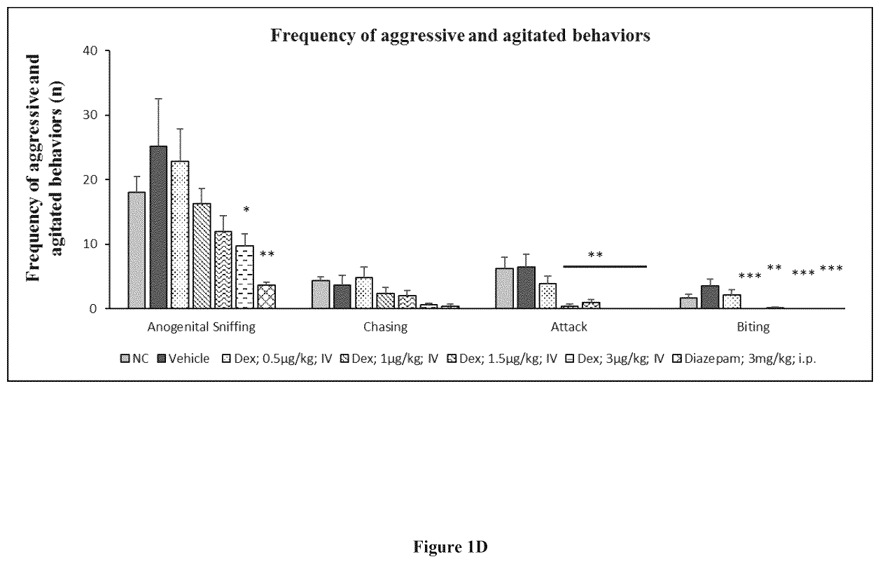

Dexmedetomidine hydrochloride (Dex) administered sublingually reduced the frequency and duration of these behaviors in a dose related manner (FIG. 1A, and FIG. 1B). Significant reduction was observed in chasing and attacking compared to vehicle control group. Similarly, intravenous administration of dexmedetomidine hydrochloride (Dex) reduced all the indices of aggressive and agitated behaviors (Figure IC and FIG. 1D). A significant reduction in anogenital sniffing, biting and attacking compared to vehicle controls was observed at doses above 0.5 μg/kg (FIG. 1C and FIG. 1D). Reference compound diazepam (3 mg/kg, i.p) also produced significant reduction in all the indices of aggressive and agitated behaviors evaluated in this study (FIG. 1A-1D).

### Effect of Sublingually/Intravenously Administered Dexmedetomidine Hydrochloride on Latency to Attack

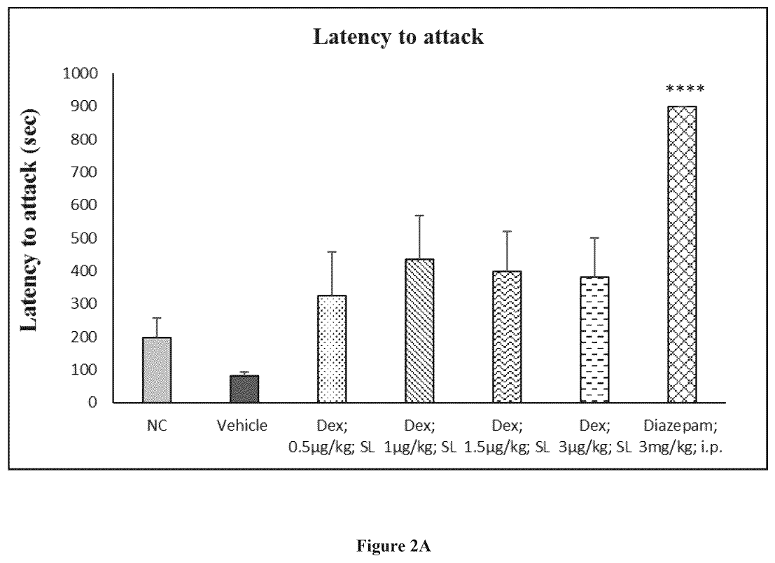

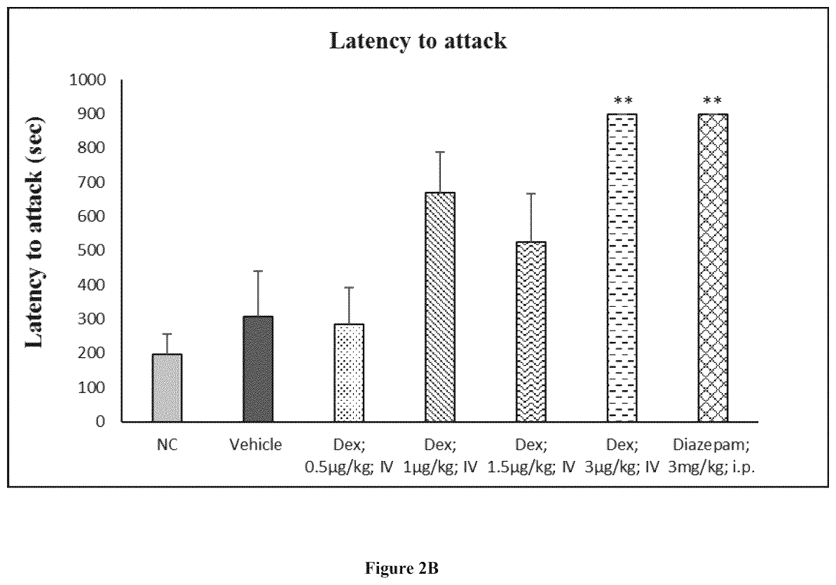

In addition to the change in frequency and duration of attack by the resident male, we also evaluated the effect of Dexmedetomidine hydrochloride (Dex) on the latency to attack the intruder rat. We observed an increase in the latency to attack the intruder rat following sublingual administration of Dexmedetomidine hydrochloride (Dex) in a dose related fashion indicating a reduction in aggression and agitation (FIG. 2A). When Dexmedetomidine hydrochloride (Dex) was administered intravenously, a similar increase in the latency to attack the intruder rat occurred in a dose related fashion that was significant compared to vehicle controls at a dose of 3 μg/kg (FIG. 2B). Animals treated with diazepam demonstrated a complete lack of attacking behavior (FIG. 2A and 2B).

### Effect of Sublingually/Intravenously Administered Dexmedetomidine Hydrochloride on Neutral Behaviors

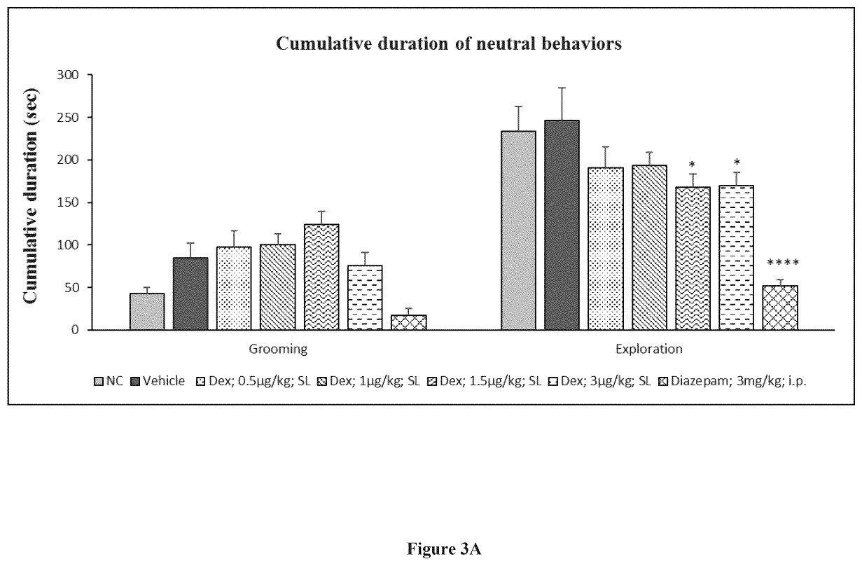

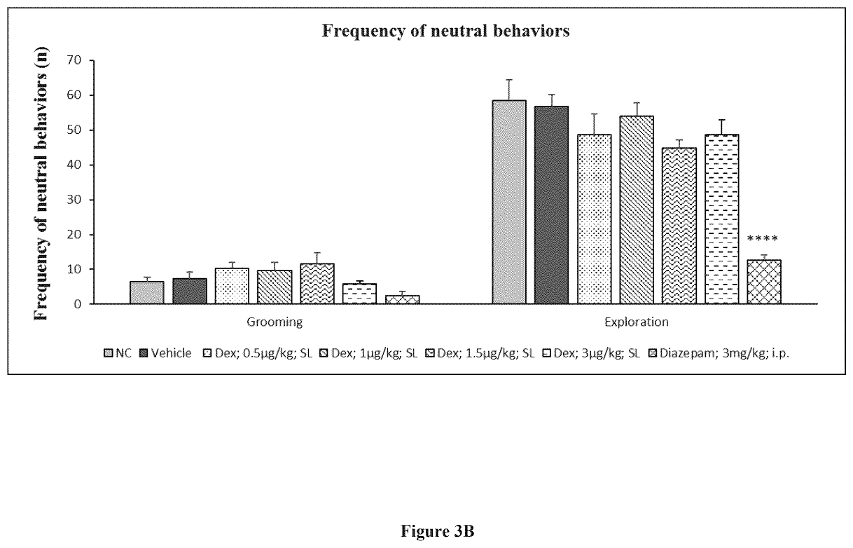

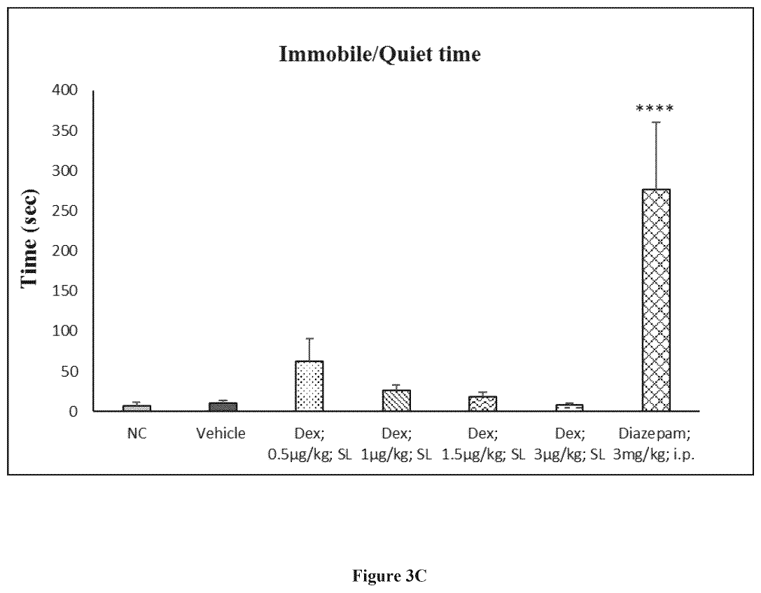

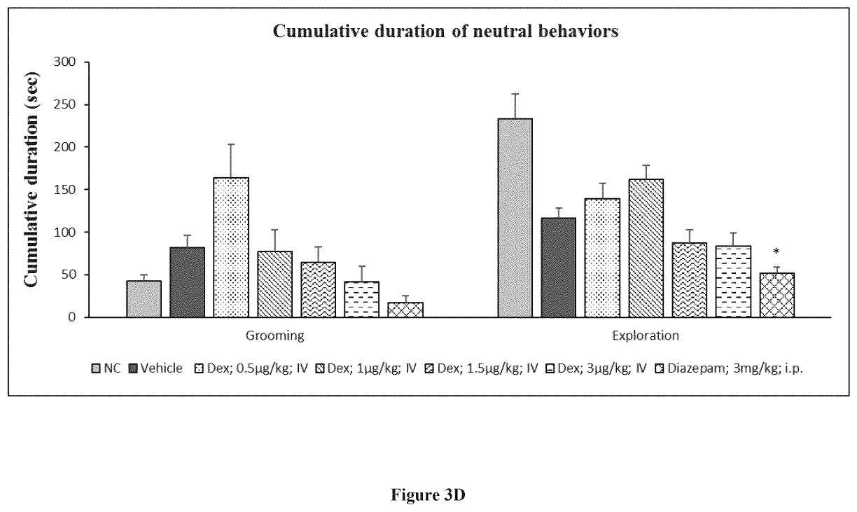

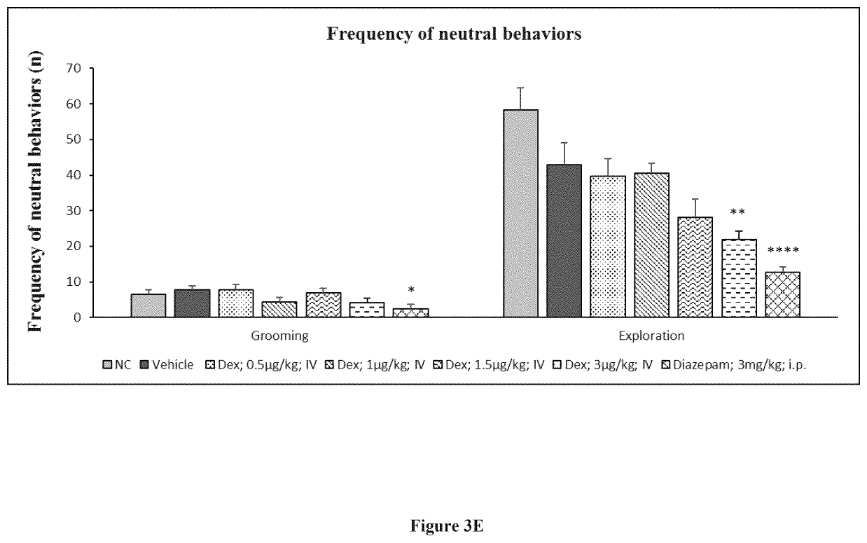

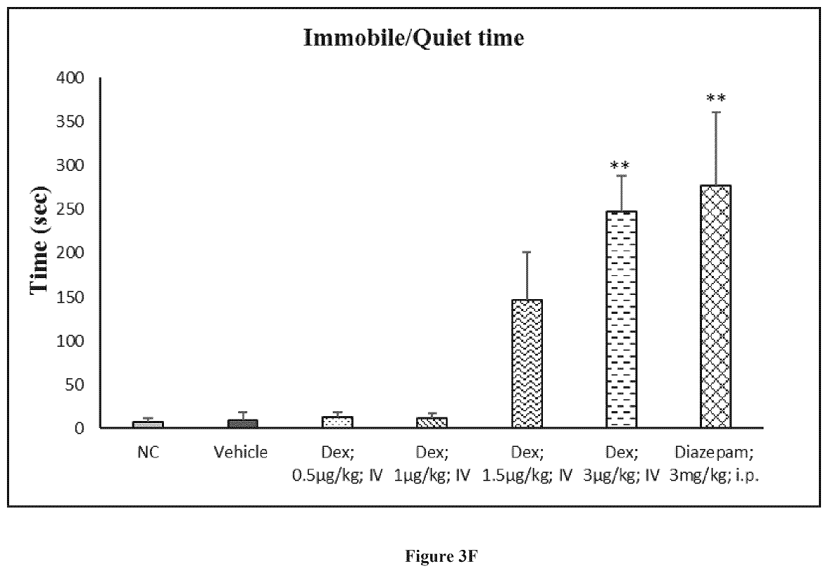

Neutral behaviors like grooming, exploration and immobile/quiet time were assessed following treatment with Dexmedetomidine hydrochloride. No significant changes occurred in the grooming and exploration following sublingual administration of Dexmedetomidine hydrochloride except a reduction in exploration observed at doses of 1.5 μg/kg & 3 μg/kg (FIG. 3A and 3B), compared to vehicle controls. Similarly, intravenously administered Dexmedetomidine hydrochloride did not significantly affect grooming and exploration in comparison to vehicle controls except at a dose of 3 μg/kg. In case of immobile/quiet time, there was no significant effect of sublingually administered Dexmedetomidine hydrochloride compared to vehicle controls however, intravenously administered Dexmedetomidine hydrochloride significantly increased the immobile/quiet time at a dose of 3 μg/kg (FIG. 3C, and FIG. 3F) Reference compound Diazepam (3 mg/kg, ip) significantly reduced the frequency and duration of all neutral behaviors evaluated in this study.

### Interpretation

In the present study, we investigated the potential of Dexmedetomidine hydrochloride in reducing aggression and agitation in rat resident-intruder model. The resident-intruder model is an established preclinical model of aggression/agitation and allows spontaneous and natural expression of both offensive aggression/agitation and defensive behavior in laboratory rodents in a semi natural laboratory setting.

1. Sublingual administration of Dexmedetomidine hydrochloride resulted in a dose related reduction in several behavioral indices of aggression and agitation such as anogenital sniffing, chasing, attacking and biting.

2. A significant increase in the latency to attack the intruder rat was observed in a dose related manner with prior treatment with Dexmedetomidine hydrochloride as compared to the vehicle control group.

3. No changes were observed in neutral behavior of animals, indicating the lack of overt anxiety-like behavior in the resident rats treated with sublingually administered Dexmedetomidine hydrochloride.

4. Of the doses that were used in the study (0.5-3 μg/kg), doses of 1-1.5 μg/kg (doses administered sublingually or intravenously) effectively reduced the behavioral indices of aggression and agitation without majorly impacting the neutral behaviors.

Conclusion: Dexmedetomidine hydrochloride effectively reduces various indices of agitation and aggression in rat resident intruder model. Dose of 1-1.5 μg/kg effectively reduced the behavioral indices of aggression and agitation without majorly impacting the neutral behaviors. In the present study the efficacy of sublingually administered Dexmedetomidine hydrochloride correlates with intravenously administered Dexmedetomidine hydrochloride at these doses (Table 6).

TABLE 6
No significant differences (i.e. similar effect via sublingual
and intravenous routes) were observed in the duration
of the behavioral indices of aggression and agitation (chasing,
biting, attack, anogenital sniffing latency to attack) when compared
between sublingual and intravenous routes of dexmedetomidine
hydrochloride administration at doses of 1 and 1.5 μg/kg.
Statistical analysis was performed using student t-test.

| | p values obtained after statistical comparison of | | | | |
|---|---|---|---|---|---|
| | sublingual vs intravenous route of administration | | | | |
| | using Student's t-test | | | | |
| | Duration (sec) | | | | |
| | | | Attack/ | Anogenital- | |
| Group | Chasing | Biting | Fighting | sniffing | Latency to attack |
| NC | 1.000 | 1.000 | 1.000 | 1.000 | 1.000 |
| Vehicle | 0.207 | 0.069 | 0.290 | 0.753 | 0.136 |
| 1 μg/kg | 0.506 | 0.102 | 0.204 | 0.090 | 0.207 |
| 1.5 μg/kg | 0.125 | 0.059 | 0.107 | 0.727 | 0.508 |

*p < 0.05, **p < 0.01 ***p < 0.001 and ****p < 0.0001 Sublingual vs intravenous routes of administration.

Based on 1-1.5 μg/kg rat efficacy doses, the human equivalent sublingual doses are calculated to be 0.161 μg/kg & 0.242 μg/kg. The total human equivalent dose for a 60-kg human would be 10 and 15 μg (https://www.fda.gov/downloads/drugs/guidances/ucm078932.pdf).

### Example 3: Estimation of Dexmedetomidine (0.5-3 μg/kg) in Rat Plasma Samples by LC-MS/MS

Objective: To estimate Dexmedetomidine levels in rat plasma samples obtained after dosing animals via intravenous and sublingual routes at doses of 0.5, 1, 1.5 and 3 μg/kg.

Blood collection: To determine the plasma concentration of dexmedetomidine, Dexmedetomidine hydrochloride was administered sublingually or intravenously in rats (n=3) at different doses (Formulation 4 adjusted to 0.5, 1, 1.5, 3 μg/kg). Blood was collected under mild isoflurane anesthesia from the retro-orbital plexus at 0, 5, 15, 30, 60 and 120 minutes post dosing. Plasma was separated and stored at −80° C. until Dexmedetomidine concentration was analyzed.

### Materials and Methods

### Preparation of Standard Solutions

A standard stock solution of dexmedetomidine hydrochloride was prepared by dissolving 1.358 mg of dexmedetomidine hydrochloride in 1358 μl of milli-Q water to achieve a concentration of 829.071 mg/ml. Working solutions of different concentrations were prepared by using diluent (methanol: water (50:50) % v/v).

Tolbutamide was used as an internal standard and its stock solution was prepared by dissolving 25 mg of tolbutamide in 1000 μl of DMSO to achieve a concentration of 25 mg/ml. Working solutions of different concentrations were prepared by using a diluent (acetonitrile: water (50:50) % v/v).

Solution preparation for SPE and chromatography: Mobile phase A (10 mm ammonium formate, pH 3.50): 0.6306 gms of ammonium formate was weighed and transferred to a 1000 ml reagent bottle. To this, 1000 ml of milli q water was added and pH of the resulting solution was adjusted to 3.5 using formic acid.

Mobile phase B: 100% acetonitrile

Diluent (methanol: water (50:50) % v/v): 50 ml of methanol was mixed with 50 ml of milli-q water. Resulting solution was used as diluent.

Wash solution: 100 μl of ammonia was mixed with 100 ml of milli q. Resulting solution was used as wash solution.

Elution solvent: 100 μl of formic acid was mixed with 100 ml of acetonitrile. Resulting solution was used as elution solvent.

Analytical Methods: Samples were analysed by using Agilent 1290 Infinity II HPLC system coupled to AB Sciex Triple Quad instrument (API-5000). Chromatographic separation was done using Agilent Zorbax Eclipse plus C18 column (50*2.1 mm, 1.8 μm) in gradient mode. The mobile phase consisted of 10 mM Ammonium Formate with pH 3.5 (Mobile phase A) and 100% Acetonitrile (Mobile phase B). The column temperature was 40° C. and flow rate was 0.35 mL/min. The MS instrument was operated in the positive mode (ESI+). For analysis, 2 μL of sample was injected into the LC-MS/MS instrument. Auto sampler temperature was 7° C.

Quality control (QC) samples were prepared as following as per table 7:

TABLE 7

| | Volume | | | | |
|---|---|---|---|---|---|
| Dexmedetomidine | of | Blank | Total | Final | |
| conc (Solution A) | solution | plasma | Volume | Calibration | QC |
| (ng/mL) | A (μL) | (μL) | (μL) | Conc (pg/mL) | ID |
| 1.114 | 2 | 48 | 50 | 44.571 | LQC |
| 371.424 | 2 | 48 | 50 | 14856.962 | MQC |
| 928.560 | 2 | 48 | 50 | 37142.406 | HQC |

### Sample Preparation

WCX SPE 96 well plate was used for sample preparation. 50 μl of plasma sample was used for extraction. Along with study samples, one set of linearity and two sets of quality controls (QC) were also processed.

Sample pretreatment: To 50 μl of plasma, 10 μl of tolbutamide working solution was added (Tolbutamide 250 ng/ml). After mixing, 50 μL of buffer solution (10 mM Ammonium Formate pH 3.5) was added. Contents were vortex mixed and loaded to preconditioned SPE plate.

### LC-MS/MS ANALYSIS

After placing the cartridges in the negative pressure SPE unit, they were conditioned by passing 200 μl of 100% methanol followed by 200 μl of water. The pretreated plasma samples were then loaded to the pre-conditioned cartridges.

After loading pretreated plasma samples, cartridges were washed with 100 μl of 0.1% ammonia solution. Finally, bound analyte was eluted with 50 μl of 0.1% formic acid in acetonitrile. This step was repeated twice for complete elution. Final eluent volume was 100 μL. To 100 μL of eluent, 50 μL of 10 mM ammonium formate (pH 3.5) was added samples were vortex mixed and transferred to a 96-well HPLC sample plate (Agilent) and submitted for LC-MS/MS analysis. For LC-MS/MS analysis, 2 μL of sample was injected. Calibration standards and QCs were processed the same way as done for study samples.

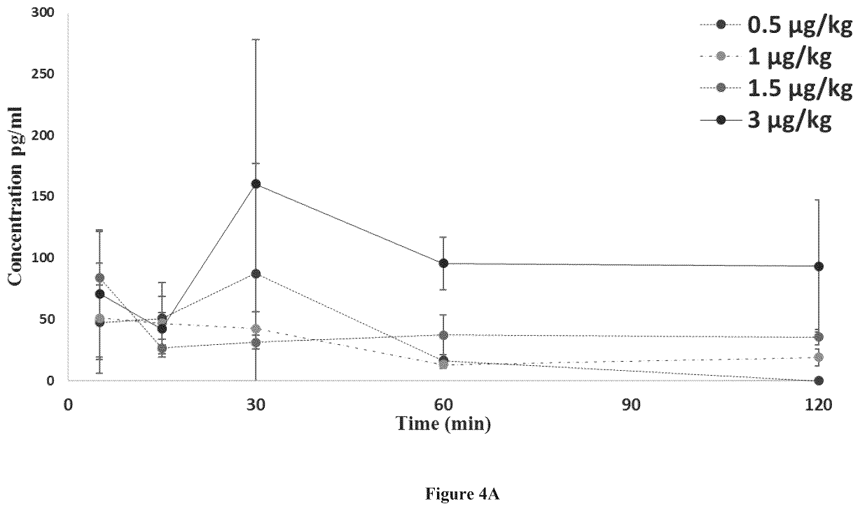

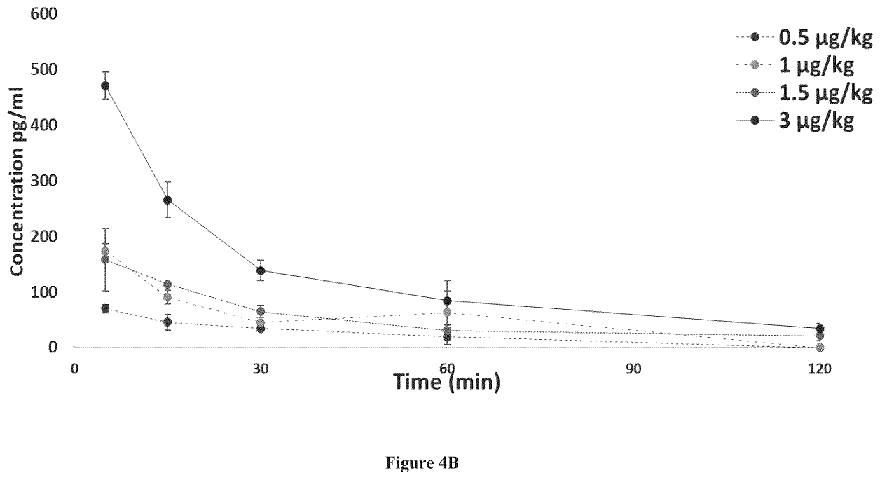

Mean plasma concentrations of Dexmedetomidine in various rat plasma samples at various time points was determined by LC-MS/MS method using Analyst 1.6.2 software (Table 8 and FIGS. 4A and 4B) with a calibration curve in the range of 0.011-53.061 ng/ml prepared in blank rat plasma matrix. The calibration curve was fitted by linear regression. The concentrations in the QC and test samples (pg/mL) were obtained from the Analyst software based on the calibration curve. Acceptance criteria for the calibration curve and QCs are as follows: 1) At least 75% of the non-zero calibration standards must be included in the calibration curve with all back-calculated concentrations within ±20% deviation from nominal concentrations (except for the lower level of quantification, LLOQ, where ±20% deviation is acceptable). 2) The correlation coefficient (r) of the calibration curve must be greater than or equal to 0.99. 3) At least two-thirds (4 out of 6) QC samples must be within ±20% relative error (accuracy)

### Results

TABLE 8
Mean rat plasma concentrations following Sublingual or Intravenous
dexmedetomidine hydrochloride administration at varying doses

| | | | | | | | Intravenous | | | | | | |
|---|---|---|---|---|---|---|---|---|---|---|---|---|---|
| Sublingual | Mean Concentration in pg/mL at | | | | | | dosing | Concentration in pg/mL at various time | | | | | |
| Dosing | various time points after dosing | | | | | | groups | points after dosing | | | | | |
| groups (I-IV) | 0 min | 5 min | 15 min | 30 min | 60 min | 120 min | (V-VIII) | 0 min | 5 min | 15 min | 30 min | 60 min | 120 min |
| I-Dex. HCl | BLQ | 48 ± | 51 ± | 87 ± | 17 ± | BLQ | V- | BLQ | 70 ± | 46 ± | 35 ± | 19 ± | BLQ |
| 0.5 μg/kg, SL | | 30.4 | 29.1 | 89.7 | 0.7 | | Dex. HCl | | 7.2 | 14.2 | 4.9 | 3.5 | |
| | | | | | | | 0.5 μg/kg, | | | | | | |
| | | | | | | | i.v. | | | | | | |
| II-Dex. HCl | BLQ | 51 ± | 47 ± | 43 ± | 13 ± | 19 ± | VI- | BLQ | 174 ± | 90 ± | 45 ± | 63 ± | BLQ |
| 1 μg/kg, SL | | 44.7 | 22.4 | 13.5 | 2.8 | 7.07 | Dex. HCl | | 12.5 | 12.1 | 1.7 | 58.0 | |
| | | | | | | | 1 μg/kg, | | | | | | |
| | | | | | | | i.v. | | | | | | |
| III-Dex. HCl | BLQ | 84 ± | 27 ± | 31 ± | 37 ± | 36 ± | VII- | BLQ | 158 ± | 114 ± | 65 ± | 31 ± | 21 ± |
| 1.5 μg/kg, SL | | 37.7 | 7.1 | 5.5 | 16.3 | 6.36 | Dex. HCl | | 56.1 | 1.7 | 11.0 | 10.3 | 8.89 |
| | | | | | | | 1.5 μg/kg, | | | | | | |
| | | | | | | | i.v. | | | | | | |
| IV-Dex. HCl | BLQ | 71 ± | 42 ± | 160 ± | 96 ± | 93 ± | VIII- | BLQ | 471 ± | 266 ± | 139 ± | 84 ± | 34 ± |
| 3 μg/kg, SL | | 52.0 | 13.0 | 117.9 | 21.5 | 53.95 | Dex. HCl | | 24.9 | 31.6 | 18.0 | 17.4 | 9.61 |
| | | | | | | | 3 μg/kg | | | | | | |
| | | | | | | | i.v. | | | | | | |

BLQ: Below the Lowest limit of Quantification of the assay (LOQ: 0.05 ng/ml)
SL: sublingual;
i.v.: intravenous
Data expressed as Mean ± SD

### Interpretation and Conclusion

Following sublingual administration of Dexmedetomidine hydrochloride, a dose-related effect on plasma concentrations was observed at doses ranging from 0.5-3 μg/kg (FIG. 4A, table 8).

Following intravenous administration of Dexmedetomidine hydrochloride, a dose-dependent effect on plasma concentrations was observed at doses ranging from 0.5-3 μg/kg (FIG. 4B, table 8).

Doses of 1 and 1.5 μg/kg effectively reduced various indices of agitation and aggression without majorly impacting neutral behaviors. Plasma concentrations following administration of dose of 1 μg/kg (via sublingual and intravenous route) between 15 to 30 min (time corresponding to the time of behavioral response observed in the efficacy study; drug administered 15 min prior to agitation behavior test & animal observed for 15 min) range from 43±13.5 to 90±12.1 pg/ml (Table 8). Similarly, plasma concentrations following administration of dose of 1.5 μg/kg (via sublingual and intravenous route) between 15 to 30 min range from 27±7.1-114 ±1.7 pg/ml (Table 8).

What is claimed is:

1. A method of treating agitation associated with dementia due to Alzheimer's Disease in an agitated subject, consisting of administering to the oral mucosa of said agitated subject one or two single dosage administrations of dexmedetomidine or a pharmaceutically acceptable salt thereof without also causing significant sedation after the administration, wherein the effective amount of dexmedetomidine or the pharmaceutically acceptable salt thereof produces an anti-agitation effect in less than about 60 minutes after administration, wherein each of the one or two single dosages of dexmedetomidine or a pharmaceutically acceptable salt thereof is 60 micrograms; wherein the agitation is not perioperative agitation; and wherein the dexmedetomidine or a pharmaceutically acceptable salt thereof is administered in a solid, water-soluble dosage form.

2. The method according to claim 1, wherein said dexmedetomidine or a pharmaceutically acceptable salt thereof is administered in a dosage form selected from the group consisting of a film, a wafer, and a tablet.

3. The method according to claim 1, wherein the agitation is acute agitation.

4. The method according to claim 1, wherein the agitation is chronic agitation.

5. The method according to claim 1, wherein the agitation is severe agitation.

6. The method according to claim 1, wherein the dexmedetomidine or a pharmaceutically acceptable salt thereof is administered sublingually.

7. The method according to claim 6, wherein the dexmedetomidine or a pharmaceutically acceptable salt thereof is administered sublingually as a film.

8. The method according to claim 7, wherein the dexmedetomidine or a pharmaceutically acceptable salt thereof is dexmedetomidine hydrochloride.

9. The method according to claim 1, wherein the dexmedetomidine or a pharmaceutically acceptable salt thereof is administered buccally.

10. The method according to claim 9, wherein the dexmedetomidine or a pharmaceutically acceptable salt thereof is administered buccally as a film.

11. The method according to claim 10, wherein the dexmedetomidine or a pharmaceutically acceptable salt thereof is dexmedetomidine hydrochloride.

12. A method of treating agitation associated with dementia due to Alzheimer's Disease in an agitated subject, consisting of administering to the oral mucosa of said agitated subject one or two single dosage administrations of dexmedetomidine or a pharmaceutically acceptable salt thereof without causing significant sedation after the administration, wherein dexmedetomidine or a pharmaceutically acceptable salt thereof produces an anti-agitation effect in less than about 60 minutes after administration, wherein each of the one or two single dosages of dexmedetomidine or a pharmaceutically acceptable salt thereof is administered at a dosage of 60 micrograms; wherein the agitation is not perioperative agitation; and wherein the dexmedetomidine or a pharmaceutically acceptable salt thereof is a film.

13. The method according to claim 12, wherein the film is administered sublingually.

14. The method according to claim 12, wherein the film is administered buccally.
